(19) United States
(12) Patent Application Publication
(10) Pub. No.: US 2008/0213878 A1
(43) Pub. Date: Sep. 4, 2008

(54) Novel human membrane proteins and polynucleotides encoding the same

(76) Inventors: Gregory Donoho, Indianapolis, IN (US); Carl Johan Friddle, The Woodlands, TX (US); Glenn Friedrich, Houston, TX (US); Brenda Gerhardt, Spring, TX (US); Erin Hilbun, Denton, TX (US); Yi Hu, Spring, TX (US); Brian Mathur, Nashville, TN (US); Maricar Miranda, The Woodlands, TX (US); Michael C. Nehls, Stockdorf (DE); Boris Nepomnichy, Dickinson, TX (US); Arthur T. Sands, The Woodlands, TX (US); John Scoville, Pearland, TX (US); Hong Sun, Bellevue, WA (US); C. Alexander Turner, The Woodlands, TX (US); D. Wade Walke, Spring, TX (US); Xiaoming Wang, Burr Ridge, IL (US); Nathaniel L. Wilganowski, Spring, TX (US); Xuanchuan Yu, Conroe, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 11/365,756

(22) Filed: Mar. 1, 2006

Related U.S. Application Data
(63) Continuation-in-part of application No. 10/813,588, filed on Mar. 30, 2004, now abandoned.
Application No. 10/813,588 is a continuation of application No. 09/691,344, filed on Oct. 18, 2000, now Pat. No. 6,743,907.
Application No. 09/691,344 is a continuation-in-part of application No. 10/941,305, filed on Sep. 15, 2004, now abandoned.
Application No. 10/941,305 is a continuation of application No. 09/813,290, filed on Mar. 20, 2001, now Pat. No. 6,815,538.
Application No. 09/813,290 is a continuation-in-part of application No. 10/980,986, filed on Nov. 4, 2004, now abandoned.
Application No. 10/980,986 is a continuation of application No. 09/818,990, filed on Mar. 27, 2001, now abandoned.
Application No. 09/818,990 is a continuation-in-part of application No. 10/833,509, filed on Apr. 28, 2004, now Pat. No. 7,199,221.
Application No. 10/833,509 is a continuation of application No. 09/854,845, filed on May 14, 2001, now Pat. No. 6,750,054.
Application No. 09/854,845 is a continuation-in-part of application No. 10/090,427, filed on Feb. 28, 2002, now abandoned.
Application No. 10/090,427 is a continuation-in-part of application No. 10/919,053, filed on Aug. 16, 2004, now abandoned.
Application No. 10/919,053 is a continuation of application No. 10/090,516, filed on Mar. 1, 2002, now abandoned.
Application No. 10/090,516 is a continuation-in-part of application No. 10/972,983, filed on Oct. 25, 2004, now abandoned.
Application No. 10/972,983 is a continuation of application No. 10/092,390, filed on Mar. 6, 2002, now abandoned.
Application No. 10/092,390 is a continuation-in-part of application No. 10/895,162, filed on Jul. 20, 2004, now abandoned.
Application No. 10/895,162 is a continuation of application No. 10/109,528, filed on Mar. 27, 2002, now abandoned.
Application No. 10/109,528 is a continuation-in-part of application No. 11/194,496, filed on Aug. 1, 2005, now abandoned.
Application No. 11/194,496 is a continuation of application No. 10/139,136, filed on May 3, 2002, now abandoned.
Application No. 10/139,136 is a continuation-in-part of application No. 11/195,579, filed on Aug. 2, 2005, now abandoned.
Application No. 11/195,579 is a continuation of application No. 10/141,670, filed on May 7, 2002, now abandoned.
Application No. 10/141,670 is a continuation-in-part of application No. 10/141,260, filed on May 7, 2002, now abandoned.
Application No. 10/141,260 is a continuation-in-part of application No. 11/260,505, filed on Oct. 27, 2005, now abandoned.
Application No. 11/260,505 is a continuation of application No. 10/180,477, filed on Jun. 25, 2002, now abandoned.
Application No. 10/180,477 is a continuation-in-part of application No. 11/177,675, filed on Jul. 8, 2005, now abandoned.
Application No. 11/177,675 is a continuation of application No. 10/225,566, filed on Aug. 21, 2002, now abandoned.
Application No. 10/225,566 is a continuation-in-part of application No. 10/962,211, filed on Oct. 8, 2004, now abandoned.
Application No. 10/962,211 is a continuation of application No. 10/225,565, filed on Aug. 21, 2002, now abandoned.
(60) Provisional application No. 60/183,583, filed on Feb. 18, 2000.
(60) Provisional application No. 60/160,285, filed on Oct. 19, 1999.
(60) Provisional application No. 60/193,639, filed on Mar. 31, 2000.
(60) Provisional application No. 60/191,188, filed on Mar. 22, 2000.
(60) Provisional application No. 60/190,638, filed on Mar. 20, 2000.
(60) Provisional application No. 60/192,218, filed on Mar. 27, 2000.
(60) Provisional application No. 60/208,893, filed on Jun. 2, 2000.
(60) Provisional application No. 60/205,274, filed on May 18, 2000.
(60) Provisional application No. 60/274,963, filed on Mar. 12, 2001.
(60) Provisional application No. 60/275,011, filed on Mar. 12, 2001.
(60) Provisional application No. 60/275,013, filed on Mar. 12, 2001.
(60) Provisional application No. 60/279,161, filed on Mar. 27, 2001.
(60) Provisional application No. 60/288,541, filed on May 3, 2001.
(60) Provisional application No. 60/289,726, filed on May 9, 2001.
(60) Provisional application No. 60/289,423, filed on May 8, 2001.
(60) Provisional application No. 60/289,424, filed on May 8, 2001.
(60) Provisional application No. 60/301,606, filed on Jun. 28, 2001.
(60) Provisional application No. 60/313,877, filed on Aug. 21, 2001.
(60) Provisional application No. 60/314,052, filed on Aug. 22, 2001.

(51) Int. Cl. C12N 15/00; C12N 15/11; C07K 14/00; C07K 16/18

(52) U.S. Cl. 435/320.1; 536/23.5; 530/350; 530/387.9

(57) ABSTRACT

Novel human polynucleotide and polypeptide sequences are disclosed that can be used in therapeutic, diagnostic, and pharmacogenomic applications.

### 1.0 CROSS-REFERENCE TO RELATED APPLICATIONS

The present application is a continuation-in-part of: co-pending U.S. application Ser. No. 10/813,588, filed on Mar. 30, 2004, which is a continuation of U.S. application Ser. No. 09/691,344, filed on Oct. 18, 2000, which issued as U.S. Pat. No. 6,743,907 B1 on Jun. 1, 2004, which claims the benefit of U.S. Provisional Application Nos. 60/183,583, filed on Feb. 18, 2000, and 60/160,285, filed on Oct. 19, 1999; co-pending U.S. application Ser. No. 10/941,305, filed on Sep. 15, 2004, which is a continuation of U.S. application Ser. No. 09/813,290, filed on Mar. 20, 2001, which issued as U.S. Pat. No. 6,815,538 B2 on Nov. 9, 2004, which claims the benefit of U.S. Provisional Application Nos. 60/193,639, filed on Mar. 31, 2000, 60/191,188, filed on Mar. 22, 2000, and 60/190,638, filed on Mar. 20, 2000; co-pending U.S. application Ser. No. 10/980,986, filed on Nov. 4, 2004, which is a continuation of U.S. application Ser. No. 09/818,990, filed on Mar. 27, 2001, abandoned, which claims the benefit of U.S. Provisional Application No. 60/192,218, filed on Mar. 27, 2000; co-pending U.S. application Ser. No. 10/833,509, filed on Apr. 28, 2004, which is a continuation of U.S. application Ser. No. 09/854,845, filed on May 14, 2001, which issued as U.S. Pat. No. 6,750,054 B2 on Jun. 15, 2004, which claims the benefit of U.S. Provisional Application Nos. 60/208,893, filed on Jun. 2, 2000, and 60/205,274, filed on May 18, 2000; co-pending U.S. application Ser. No. 10/090,427, filed on Feb. 28, 2002, which claims the benefit of U.S. Provisional Application No. 60/274,963, filed on Mar. 12, 2001; co-pending U.S. Application Serial Number 10/919,053, filed on Aug. 16, 2004, which is a continuation of U.S. application Ser. No. 10/090,516, filed on Mar. 1, 2002, abandoned, which claims the benefit of U.S. Provisional Application No. 60/275,011, filed on Mar. 12, 2001; co-pending U.S. application Ser. No. 10/972,983, filed on Oct. 25, 2004, which is a continuation of U.S. application Ser. No. 10/092,390, filed on Mar. 6, 2002, abandoned, which claims the benefit of U.S. Provisional Application No. 60/275,013, filed on Mar. 12, 2001; co-pending U.S. application Ser. No. 10/895,162, filed on Jul. 20, 2004, which is a continuation of U.S. application Ser. No. 10/109,528, filed on Mar. 27, 2002, abandoned, which claims the benefit of U.S. Provisional Application No. 60/279,161, filed on Mar. 27, 2001; co-pending U.S. application Ser. No. 11/194,496, filed on Aug. 1, 2005, which is a continuation of U.S. application Ser. No. 10/139,136, filed on May 3, 2002, abandoned, which claims the benefit of U.S. Provisional Application No. 60/288,541, filed on May 3, 2001; co-pending U.S. application Ser. No. 11/195,579, filed on Aug. 2, 2005, which is a continuation of U.S. application Ser. No. 10/141,670, filed on May 7, 2002, abandoned, which claims the benefit of U.S. Provisional Application Nos. 60/289,726, filed on May 9, 2001, and 60/289,423, filed on May 8, 2001; co-pending U.S. application Ser. No. 10/141,260, filed on May 7, 2002, which claims the benefit of U.S. Provisional Application No. 60/289,424, filed on May 8, 2001; co-pending U.S. application Ser. No. 11/260,505, filed on Oct. 27, 2005, which is a continuation of U.S. application Ser. No. 10/180,477, filed on Jun. 25, 2002, abandoned, which claims the benefit of U.S. Provisional Application No. 60/301,606, filed on Jun. 28, 2001; co-pending U.S. application Ser. No. 11/177,675, filed on Jul. 8, 2005, which is a continuation of U.S. application Ser. No. 10/225,566, filed on Aug. 21, 2002, abandoned, which claims the benefit of U.S. Provisional Application No. 60/313,877, filed on Aug. 21, 2001; and co-pending U.S. application Ser. No. 10/962,211, filed on Oct. 8, 2004, which is a continuation of U.S. application Ser. No. 10/225,565, filed on Aug. 21, 2002, abandoned, which claims the benefit of U.S. Provisional Application No. 60/314,052, filed on Aug. 22, 2001; each of which is herein incorporated by reference in its entirety.

### 2.0 CROSS-REFERENCE TO SEQUENCE LISTING SUBMITTED ON COMPACT DISC

The present application contains a Sequence Listing of SEQ ID NOS:1-163, in file “FINALSeqList.txt” (1,492,992 bytes), created on Mar. 1, 2006, submitted herewith on duplicate compact disc (Copy 1 and Copy 2), which is herein incorporated by reference in its entirety.

### 3.0 INTRODUCTION

The present invention relates to the discovery, identification, and characterization of novel human polynucleotides encoding proteins that share sequence similarity with mammalian membrane and secreted proteins. The invention encompasses the described polynucleotides, host cell expression systems, the encoded proteins, fusion proteins, polypeptides and peptides, antibodies to the encoded proteins and peptides, and genetically engineered animals that either lack or overexpress the disclosed polynucleotides, antagonists and agonists of the proteins, and other compounds that modulate the expression or activity of the proteins encoded by the disclosed polynucleotides, which can be used for diagnosis, drug screening, clinical trial monitoring, the treatment of diseases and disorders, and cosmetic or nutriceutical applications.

### 4.0 BACKGROUND OF THE INVENTION

In addition to providing the structural and mechanical scaffolding for cells and tissues, membrane proteins can also serve as recognition markers, mediate signal transduction, mediate or facilitate the passage of materials across the lipid bilayer, and can act as adhesion proteins that facilitate tissue organization, development, and integrity.

The CUB domain is an extracellular domain (ECD) present in variety of diverse proteins such as bone morphogenetic protein 1, proteinases, spermadhesins, complement subcomponents, and neuronal recognition molecules. Given the importance of these functions, CUB proteins have been associated with, inter alia, regulating development, modulating cellular processes, modulating ligand-receptor interactions, and preventing infectious disease. Ig-domain proteins are typically membrane-associated proteins that bind receptors and ligands, and can participate in signal transduction.

Secreted proteins are biologically active molecules that have been implicated in a number of biological processes and anomalies such as hyperproliferative disorders, muscle contraction, vasoconstriction and dilation, immunity, development, modulating metabolism, and cancer. In particular, protein hormones have been implicated in, inter alia, autoimmunity, diabetes, osteoporosis, infectious disease, arthritis, and modulating physiological homeostasis, metabolism, and behavior. Examples of biologically active secreted proteins include, but are not limited to, semaphorins which have been implicated in, inter alia, mediating neural processes, axon guidance, cancer, and development. Along with their cognate receptors (i.e., neuropilins or plexins), semaphorins act to regulate the organization and fasciculation of nerves in the body.

### 5.0 SUMMARY OF THE INVENTION

The present invention relates to the discovery, identification, and characterization of nucleotides that encode novel human proteins, and the corresponding amino acid sequences of these proteins. The novel human proteins, described for the first time herein, share structural similarity with: animal CUB domain proteins, coagulation factors V and XIII, milk fat globule-EGF factor 8, transcriptional repressor AE-binding protein-1, and neuropilins 1 and 2, which contain both CUB and discoidin domains (SEQ ID NOS:1-7); animal secreted proteins such as semaphorin and collapsin proteins (SEQ ID NOS: 8-12); animal secreted proteins such as protein/peptide hormones of the neurohypophysial family (SEQ ID NOS:13 and 14); animal secreted proteins such as protein/peptide hormones of the oxytocin (neurophysin 1 precursor) family (SEQ ID NOS:15-17); mammalian muscle proteins (myosin light chain kinase, telokin, IgG like C2 domains, myotilin), modifiers and anchors thereof, and sequences that are similar to various domains (i.e., kinase, Ig, adhesin, etc.) within the mammalian titin protein (SEQ ID NOS:18-45); mammalian semaphorin proteins, particularly semaphorin G and F (SEQ ID NOS:46-62); mammalian semaphorin proteins, particularly semaphorin G (subclass 4) and semaphorin F (SEQ ID NOS:63-95); mammalian semaphorin proteins, particularly semaphorin 6A1 (SEQ ID NOS:96-108); mammalian membrane proteins such as the murine TEN-M4/cdz protein (a dimeric type II transmembrane protein that apparently represents the murine ortholog of the described sequences), and proteins identified as gamma-heregulins (SEQ ID NOS:109-112); mammalian membrane proteins of the epidermal growth factor (EGF) family, and notch proteins (SEQ ID NOS:113-116); mammalian adhesin and cell polarity proteins, and proteins having structural domains in common with proteins associated with cell adhesion and/or tissue organization and integrity (SEQ ID NOS:117-125); mammalian proteins having structural domains in common with proteins of the immunoglobulin (Ig) super family, which are often found on the cell surface and can be used in signaling pathways, receptor-mediated interactions, and can be exploited by human pathogens to gain entry to the cell, and more particularly, the murine Punc gene (SEQ ID NOS:126 and 127); mammalian proteins having structural domains in common with proteins of the immunoglobulin (Ig) super family (SEQ ID NOS:128 and 129); mammalian proteins having structural domains in common with laminin G proteins, thrombospondins, and proteins of the cadherin and protocadherin family (SEQ ID NOS:130-132); proteins of the MUNC family, which have been implicated in neural development and function (SEQ ID NOS:133-136); mammalian semaphorin receptors, and particularly plexins (SEQ ID NOS:137-148); mammalian proteins having structural domains in common with proteins of the immunoglobulin (Ig) super family, and particularly, human immunoglobulin receptors (SEQ ID NOS:149-156); mammalian glycoproteins, secreted proteins, and receptors, particularly proteins characterized by the presence of at least one Ig-domain, such as CEA-related cell adhesion molecule, mouse hepatitis virus receptor, biliary glycoprotein 1, carcinoembryonic antigen 7 and 1, and Rig-1, see GenBank Accession Number XM_{—}166754 (SEQ ID NOS:157 and 158); and animal CUB domain and “sushi” domain proteins, and proteins such as vascular endothelial cell growth factor and neuropilins (SEQ ID NOS:159-163).

The novel human nucleic acid sequences described herein (SEQ ID NOS:1, 3, 5, 8, 10, 13, 15, 18, 20, 22, 24, 26, 28, 30, 32, 34, 36, 38, 40, 42, 44, 46, 48, 50, 52, 54, 56, 58, 60, 63, 65, 67, 69, 71, 73, 75, 77, 79, 81, 83, 85, 87, 89, 91, 93, 96, 98, 100, 102, 104, 106, 109, 111, 113, 115, 117, 119, 122, 124, 126, 128, 130, 133, 135, 137, 139, 141, 143, 145, 147, 149, 151, 154, 157, 159, and 161) encode proteins/open reading frames (ORFs) of 487, 586, 539, 875, 782, 91, 89, 1320, 376, 419, 401, 459, 570, 754, 1045, 102, 144, 126, 184, 295, 479, 1049, 1093, 1034, 1078, 1151, 1136, 954, 939, 215, 496, 702, 697, 843, 838, 870, 865, 116, 397, 603, 598, 744, 739, 771, 766, 1073, 998, 476, 1060, 985, 463, 2753, 1624, 1140, 586, 1285, 1012, 955, 448, 814, 2623, 3846, 2214, 1703, 2013, 1948, 1959, 1894, 1974, 1909, 776, 221, 666, 416, 301, and 185 amino acids in length (SEQ ID NOS:2, 4, 6, 9, 11, 14, 16, 19, 21, 23, 25, 27, 29, 31, 33, 35, 37, 39, 41, 43, 45, 47, 49, 51, 53, 55, 57, 59, 61, 64, 66, 68, 70, 72, 74, 76, 78, 80, 82, 84, 86, 88, 90, 92, 94, 97, 99, 101, 103, 105, 107, 110, 112, 114, 116, 118, 120, 123, 125, 127, 129, 131, 134, 136, 138, 140, 142, 144, 146, 148, 150, 152, 155, 158, 160, and 162). SEQ ID NOS:7, 12, 17, 62, 95, 108, 121, 132, 153, 156, and 163 describe full length ORFs, as well as flanking 5′ and 3′ sequences.

The invention also encompasses agonists and antagonists of the described proteins, including small molecules, large molecules, mutant proteins, or portions thereof, that compete with native proteins, peptides, and antibodies, as well as nucleotide sequences that can be used to inhibit the expression of the described proteins (e.g., antisense and ribozyme molecules, and open reading frame or regulatory sequence replacement constructs) or to enhance the expression of the described proteins (e.g., expression constructs that place the described polynucleotide under the control of a strong promoter system), and transgenic animals that express a described protein sequence, or “knock-outs” (which can be conditional) that do not express a functional protein. Knock-out mice can be produced in several ways, one of which involves the use of mouse embryonic stem cell (“ES cell”) lines that contain gene trap mutations in a murine homolog of at least one of the described proteins. When the unique sequences described in SEQ ID NOS:1-163 are “knocked-out” they provide a method of identifying phenotypic expression of the particular gene, as well as a method of assigning function to previously unknown genes. In addition, animals in which the unique sequences described in SEQ ID NOS:1-163 are “knocked-out” provide an unique source in which to elicit antibodies to homologous and orthologous proteins, which would have been previously viewed by the immune system as “self” and therefore would have failed to elicit significant antibody responses. To these ends, gene trapped knock-out ES cells have been generated in murine homologs of a number of the described membrane proteins.

Additionally, the unique sequences described in SEQ ID NOS:1-163 are useful for the identification of protein coding sequences, and mapping an unique gene to a particular chromosome. These sequences identify biologically verified exon splice junctions, as opposed to splice junctions that may have been bioinformatically predicted from genomic sequence alone. The sequences of the present invention are also useful as additional DNA markers for restriction fragment length polymorphism (RFLP) analysis, and in forensic biology, particularly given the presence of multiple nucleotide polymorphisms within the described sequences.

Further, the present invention also relates to processes for identifying compounds that modulate, i.e., act as agonists or antagonists of, expression and/or activity of the described proteins that utilize purified preparations of the described sequences and/or protein products, or cells expressing the same. Such compounds can be used as therapeutic agents for the treatment of any of a wide variety of symptoms associated with biological disorders or imbalances.

### 6.0 BRIEF DESCRIPTION OF THE FIGURES

No Figures are required in the present invention.

### 7.0 DETAILED DESCRIPTION OF THE INVENTION

The sequences described for the first time herein encode novel proteins that may be expressed in, inter alia, human cell lines, and: human prostate, pituitary, fetal brain, brain, thymus, spleen, lymph node, trachea, kidney, fetal liver, thyroid, adrenal gland, salivary gland, stomach, small intestine, colon, muscle, heart, mammary gland, adipose, skin, esophagus, bladder, cervix, rectum, and testis cells (SEQ ID NOS:1-7); human fetal brain, brain, cerebellum, thymus, spleen, lymph node, kidney, uterus, adipose, esophagus, cervix, rectum, pericardium, and placenta cells (SEQ ID NOS:8-12); human fetal brain, brain, pituitary, cerebellum, spinal cord, thymus, spleen, lymph node, bone marrow, trachea, kidney, fetal liver, prostate, testis, thyroid, adrenal gland, stomach, small intestine, colon, skeletal muscle, adipose, and esophagus cells (SEQ ID NOS:13 and 14); human fetal brain, brain, cerebellum, thymus, kidney, fetal liver, prostate, skeletal muscle, esophagus, rectum, pericardium, and fetal kidney cells (SEQ ID NOS:15-17); human bone marrow, trachea, fetal liver, prostate, testis, thyroid, adrenal gland, skeletal muscle, esophagus, and pericardium cells (SEQ ID NOS:18-45); human brain, pituitary, cerebellum, kidney, prostate, testis, thyroid, adrenal gland, pancreas, salivary gland, heart, uterus, cervix, pericardium, fetal kidney and fetal lung cells (SEQ ID NOS:46-62); human brain, fetal brain, lymph node, mammary gland, pituitary gland, placenta, prostate, kidney, thyroid, and umbilical vein endothelial cells (SEQ ID NOS:63-95); human fetal brain, pituitary, cerebellum, spinal cord, lymph node, lung, kidney, prostate, thyroid, adrenal gland, small intestine, colon, skeletal muscle, uterus, placenta, mammary gland, skin, esophagus, bladder, pericardium, fetal kidney, fetal lung, 6-, 9-, and 12-week embryo, and embryonic carcinoma cells (SEQ ID NOS:96-108); human fetal brain, pituitary, spinal cord, lymph node, thyroid, adrenal gland, fetal kidney, fetal lung, and 6- and 9-week old embryo cells (SEQ ID NOS:109-112); human prostate, pituitary, skeletal muscle, fetal brain, brain, adrenal gland, and osteosarcoma cells (SEQ ID NOS:113-116); human fetal brain, cerebellum, spinal cord, lymph node, bone marrow, kidney, fetal kidney, 6- and 9-week embryo, and osteosarcoma cells (SEQ ID NOS:117 and 118); human fetal brain, brain, pituitary, spinal cord, thymus, lymph node, trachea, lung, testis, thyroid, salivary gland, stomach, small intestine, heart, uterus, mammary gland, adipose, skin, fetal kidney, fetal lung, aorta, 6- and 12-week embryo, adenocarcinoma, osteosarcoma, and embryonic carcinoma cells (SEQ ID NOS:119-121); human fetal brain, brain, pituitary, cerebellum, spinal cord, thymus, spleen, lymph node, bone marrow, trachea, lung, kidney, prostate, testis, thyroid, adrenal gland, salivary gland, stomach, small intestine, skeletal muscle, heart, uterus, placenta, mammary gland, adipose, skin, esophagus, bladder, cervix, rectum, pericardium, hypothalamus, ovary, fetal kidney, fetal lung, gall bladder, tongue, aorta, 6-, 9-, and 12-week embryo, adenocarcinoma, osteosarcoma, embryonic carcinoma, umbilical vein, and endothelial cells (SEQ ID NOS:122-125); human fetal brain, brain, cerebellum, spinal cord, thymus, lymph node, prostate, testis, thyroid, uterus, placenta, hypothalamus, fetal kidney, fetal lung, tongue, 6-, 9-, and 12-week embryo, adenocarcinoma, and embryonic carcinoma cells (SEQ ID NOS:126 and 127); human fetal brain, brain, pituitary, cerebellum, spinal cord, thymus, spleen, lymph node, bone marrow, trachea, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, pericardium, hypothalamus, ovary, fetal kidney, fetal lung, 6-, 9-, and 12-week embryo, and osteosarcoma cells (SEQ ID NOS:128 and 129); human fetal brain, brain, pituitary, cerebellum, spinal cord, thymus, spleen, lymph node, bone marrow, trachea, lung, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, pericardium, ovary, fetal kidney, fetal lung, gall bladder, tongue, aorta, 6-, 9-, and 12-week old embryo, adenocarcinoma, osteosarcoma, embryonic carcinoma, umbilical vein, and microvascular endothelial cells (SEQ ID NOS:130-132); human fetal brain, brain, cerebellum, spinal cord, thymus, lymph node, bone marrow, trachea, lung, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, rectum, pericardium, hypothalamus, ovary, fetal kidney, fetal lung, gall bladder, tongue, aorta, 6-, 9-, and 12-week old embryo, adenocarcinoma, osteosarcoma, and embryonic carcinoma cells (SEQ ID NOS:133 and 134); human fetal brain, brain, cerebellum, spinal cord, lymph node, testis, adrenal gland, uterus, hypothalamus, 6- and 9-week old embryo, and osteosarcoma cells (SEQ ID NOS:135 and 136); human fetal brain, brain, pituitary, cerebellum, spinal cord, thymus, spleen, lymph node, bone marrow, trachea, lung, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, rectum, pericardium, hypothalamus, ovary, fetal kidney, fetal lung, gall bladder, tongue, aorta, 6-, 9-, and 12-week embryo, adenocarcinoma, osteosarcoma, embryonic carcinoma, and endothelial cells (SEQ ID NOS:137-148); human spleen, lymph node, and bone marrow cells (SEQ ID NOS:149-153); human thymus, lymph node, bone marrow, trachea, thyroid, adrenal gland, stomach, lung, mammary gland, fetal kidney, and fetal lung cells (SEQ ID NOS:154-156); human fetal brain, cerebellum, spinal cord, lymph node, trachea, testis, adrenal gland, mammary gland, adipose, esophagus, hypothalamus, fetal kidney, and fetal lung cells (SEQ ID NOS:157 and 158); and human fetal brain, brain, cerebellum, spinal cord, thymus, spleen, lymph node, bone marrow, trachea, lung, kidney, fetal liver, liver, prostate, testis, thyroid, adrenal gland, pancreas, salivary gland, stomach, small intestine, colon, skeletal muscle, heart, uterus, placenta, mammary gland, adipose, skin, esophagus, bladder, cervix, rectum, pericardium, hypothalamus, ovary, fetal kidney, fetal lung, gall bladder, tongue, aorta, 6-, 9-, and 12-week embryo, adenocarcinoma, and umbilical vein endothelial cells (SEQ ID NOS:159-163).

The present invention encompasses the nucleotides presented in the Sequence Listing, host cells expressing such nucleotides, the expression products of such nucleotides, and: (a) nucleotides that encode mammalian homologs of the described polynucleotides, including the specifically described nucleic acid sequences and protein products; (b) nucleotides that encode one or more portions of the proteins that correspond to functional domains, and the polypeptide products specified by such nucleotide sequences, including, but not limited to, the novel regions of any active domain(s); (c) isolated nucleotides that encode mutant versions, engineered or naturally occurring, of the described sequences, in which all or a part of at least one domain is deleted or altered, and the polypeptide products specified by such nucleotide sequences, including, but not limited to, soluble proteins and peptides in which all or a portion of the signal (or hydrophobic transmembrane) sequence is deleted; (d) nucleotides that encode chimeric fusion proteins containing all or a portion of the described coding regions, or one of its domains (e.g., a receptor or ligand binding domain, accessory protein/self-association domain, etc.), fused to another peptide or polypeptide; or (e) therapeutic or diagnostic derivatives of the described polynucleotides, such as oligonucleotides, antisense polynucleotides, ribozymes, dsRNA, or gene therapy constructs comprising a sequence first disclosed in the Sequence Listing.

As discussed above, the present invention includes the human DNA sequences presented in the Sequence Listing (and vectors comprising the same), and additionally contemplates any nucleotide sequence encoding a contiguous open reading frame (ORF) that hybridizes to a complement of a DNA sequence presented in the Sequence Listing under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (“Current Protocols in Molecular Biology”, Vol. 1, p. 2.10.3 (Ausubel et al., eds., Green Publishing Associates, Inc., and John Wiley & Sons, Inc., New York, 1989)) and encodes a functionally equivalent expression product. Additionally contemplated are any nucleotide sequences that hybridize to the complement of a DNA sequence that encodes and expresses an amino acid sequence presented in the Sequence Listing under moderately stringent conditions, e.g., washing in 0.2×SSC/0.1% SDS at 42° C. (“Current Protocols in Molecular Biology”, supra), yet still encodes a functional equivalent of the described membrane proteins. Functional equivalents of the described membrane proteins include naturally occurring membrane proteins present in other species, and mutant membrane proteins, whether naturally occurring or engineered (by site directed mutagenesis, gene shuffling, directed evolution as described in, for example, U.S. Pat. No. 5,837,458). The invention also includes degenerate nucleic acid variants of the disclosed membrane protein polynucleotide sequences.

Additionally contemplated are polynucleotides encoding membrane protein ORFs, or their functional equivalents, encoded by polynucleotide sequences that are about 99, 95, 90, or about 85 percent similar or identical to corresponding regions of the nucleotide sequences of the Sequence Listing (as measured by BLAST sequence comparison analysis using, for example, the GCG sequence analysis package (the University of Wisconsin GCG sequence analysis package, SEQUENCHER 3.0, Gene Codes Corp., Ann Arbor, Mich.) using default settings).

The invention also includes nucleic acid molecules, preferably DNA molecules, that hybridize to, and are therefore the complements of, the described nucleotide sequences. Such hybridization conditions may be highly stringent or less highly stringent, as described herein. In instances where the nucleic acid molecules are deoxyoligonucleotides, such molecules are generally about 16 to about 100 bases long, or about 20 to about 80 bases long, or about 34 to about 45 bases long, or any variation or combination of sizes represented therein that incorporate a contiguous region of sequence first disclosed in the Sequence Listing. Such oligonucleotides can be used in conjunction with the polymerase chain reaction (PCR) to screen libraries, isolate clones, and prepare cloning and sequencing templates, etc.

Alternatively, such oligonucleotides can be used as hybridization probes for screening libraries, and assessing gene expression patterns (particularly using a microarray or high-throughput “chip” format). Additionally, a series of oligonucleotide sequences, or the complements thereof, can be used to represent all or a portion of the described nucleic acid sequences. An oligonucleotide or polynucleotide sequence first disclosed in at least a portion of one or more of the sequences of SEQ ID NOS:1-163 can be used as a hybridization probe in conjunction with a solid support matrix/substrate (resins, beads, membranes, plastics, polymers, metal or metallized substrates, crystalline or polycrystalline substrates, etc.). Of particular note are spatially addressable arrays (i.e., gene chips, microtiter plates, etc.) of oligonucleotides and polynucleotides, or corresponding oligopeptides and polypeptides, wherein at least one of the biopolymers present on the spatially addressable array comprises an oligonucleotide or polynucleotide sequence first disclosed in at least one of the sequences of SEQ ID NOS:1-163, or an amino acid sequence encoded thereby. Methods for attaching biopolymers to, or synthesizing biopolymers on, solid support matrices, and conducting binding studies thereon, are disclosed in, inter alia, U.S. Pat. Nos. 5,700,637, 5,556,752, 5,744,305, 4,631,211, 5,445,934, 5,252,743, 4,713,326, 5,424,186, and 4,689,405.

Addressable arrays comprising sequences first disclosed in SEQ ID NOS:1-163 can be used to identify and characterize the temporal and tissue specific expression of a gene. These addressable arrays incorporate oligonucleotide sequences of sufficient length to confer the required specificity, yet be within the limitations of the production technology. The length of these probes is usually within a range of between about 8 to about 2000 nucleotides. Preferably the probes consist of 60 nucleotides, and more preferably 25 nucleotides, from the sequences first disclosed in SEQ ID NOS:1-163.

For example, a series of oligonucleotide sequences, or the complements thereof, can be used in chip format to represent all or a portion of the described nucleic acid sequences. The oligonucleotides, typically between about 16 to about 40 (or any whole number within the stated range) nucleotides in length, can partially overlap each other, and/or the sequence may be represented using oligonucleotides that do not overlap. Accordingly, the described polynucleotide sequences shall typically comprise at least about two or three distinct oligonucleotide sequences of at least about 8 nucleotides in length that are each first disclosed in the described Sequence Listing. Such oligonucleotide sequences can begin at any nucleotide present within a sequence in the Sequence Listing, and proceed in either a sense (5′-to-3′) orientation vis-a-vis the described sequence or in an antisense orientation.

Microarray-based analysis allows the discovery of broad patterns of genetic activity, providing new understanding of gene functions, and generating novel and unexpected insight into transcriptional processes and biological mechanisms. The use of addressable arrays comprising sequences first disclosed in SEQ ID NOS:1-163 provides detailed information about transcriptional changes involved in a specific pathway, potentially leading to the identification of novel components, or gene functions that manifest themselves as novel phenotypes.

Probes consisting of sequences first disclosed in SEQ ID NOS:1-163 can also be used in the identification, selection, and validation of novel molecular targets for drug discovery. The use of these unique sequences permits the direct confirmation of drug targets, and recognition of drug dependent changes in gene expression that are modulated through pathways distinct from the intended target of the drug. These unique sequences therefore also have utility in defining and monitoring both drug action and toxicity.

As an example of utility, the sequences first disclosed in SEQ ID NOS:1-163 can be utilized in microarrays, or other assay formats, to screen collections of genetic material from patients who have a particular medical condition. These investigations can also be carried out using the sequences first disclosed in SEQ ID NOS:1-163 in silico, and by comparing previously collected genetic databases and the disclosed sequences using computer software known to those in the art.

Thus the sequences first disclosed in SEQ ID NOS:1-163 can be used to identify mutations associated with a particular disease, and also in diagnostic or prognostic assays.

Although the presently described sequences have been specifically described using nucleotide sequence, it should be appreciated that each of the sequences can uniquely be described using any of a wide variety of additional structural attributes, or combinations thereof. For example, a given sequence can be described by the net composition of the nucleotides present within a given region of the sequence, in conjunction with the presence of one or more specific oligonucleotide sequence(s) first disclosed in SEQ ID NOS:1-163. Alternatively, a restriction map specifying the relative positions of restriction endonuclease digestion sites, or various palindromic or other specific oligonucleotide sequences, can be used to structurally describe a given sequence. Such restriction maps, which are typically generated by widely available computer programs (e.g., the University of Wisconsin GCG sequence analysis package, SEQUENCHER 3.0, Gene Codes Corp., etc.), can optionally be used in conjunction with one or more discrete nucleotide sequence(s) present in the sequence that can be described by the relative position of the sequence relative to one or more additional sequence(s) or one or more restriction sites present in the disclosed sequence.

For oligonucleotide probes, highly stringent conditions may refer, e.g., to washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides). These nucleic acid molecules may encode or act as antisense molecules, useful, for example, in membrane protein gene regulation and/or as antisense primers in amplification reactions of membrane protein nucleic acid sequences. With respect to membrane protein gene regulation, such techniques can be used to regulate biological functions. Further, such sequences may be used as part of ribozyme and/or triple helix sequences that are also useful for membrane protein gene regulation.

Inhibitory antisense or double stranded oligonucleotides can additionally comprise at least one modified base moiety that is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N2-carboxypropyl) uracil, (acp3)w, and 2,6-diaminopurine.

The antisense oligonucleotide can also comprise at least one modified sugar moiety selected from the group including, but not limited to, arabinose, 2-fluoroarabinose, xylulose, and hexose.

In yet another embodiment, the antisense oligonucleotide will comprise at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual 1-units, the strands run parallel to each other (Gautier et al., Nucl. Acids Res. 15:6625-6641, 1987). The oligonucleotide is a 2′-1-methylribonucleotide (Inoue et al., Nucl. Acids Res. 15:6131-6148, 1987), or a chimeric RNA-DNA analogue (Inoue et al., FEBS Lett. 215:327-330, 1987). Alternatively, double stranded RNA can be used to disrupt the expression and function of a targeted membrane protein.

Oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized (Stein et al., Nucl. Acids Res. 16:3209-3221, 1988), and methylphosphonate oligonucleotides can be prepared by use of controlled pore glass polymer supports (Sarin et al., Proc. Natl. Acad. Sci. USA 85:7448-7451, 1988), etc.

Low stringency conditions are well-known to those of skill in the art, and will vary predictably depending on the specific organisms from which the library and the labeled sequences are derived. For guidance regarding such conditions, see, for example, “Molecular Cloning, A Laboratory Manual” (Sambrook et al., eds., Cold Spring Harbor Press, Cold Spring Harbor, N.Y., 1989), “Current Protocols in Molecular Biology”, supra, and periodic updates thereof.

Alternatively, suitably labeled membrane protein nucleotide probes can be used to screen a human genomic library using appropriately stringent conditions or by PCR. The identification and characterization of human genomic clones is helpful for identifying polymorphisms (including, but not limited to, nucleotide repeats, microsatellite alleles, single nucleotide polymorphisms, or coding single nucleotide polymorphisms), determining the genomic structure of a given locus/allele, and designing diagnostic tests. For example, sequences derived from regions adjacent to the intron/exon boundaries of the human gene can be used to design primers for use in amplification assays to detect mutations within the exons, introns, splice sites (e.g., splice acceptor and/or donor sites), etc., that can be used in diagnostics and pharmacogenomics.

For example, the present sequences can be used in restriction fragment length polymorphism (RFLP) analysis to identify specific individuals. In this technique, an individual's genomic DNA is digested with one or more restriction enzymes, and probed on a Southern blot to yield unique bands for identification (as generally described in U.S. Pat. No. 5,272,057). In addition, the sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). Actual base sequence information can be used for identification as an accurate alternative to patterns formed by restriction enzyme generated fragments.

Further, a membrane protein gene homolog can be isolated from nucleic acid from an organism of interest by performing PCR using two degenerate or “wobble” oligonucleotide primer pools designed on the basis of amino acid sequences within the membrane protein products disclosed herein. The template for the reaction may be genomic DNA, or total RNA, mRNA, and/or cDNA obtained by reverse transcription of mRNA prepared from human or non-human cell lines or tissue known to express, or suspected of expressing, an allele of a membrane protein gene. The PCR product can be subcloned and sequenced to ensure that the amplified sequences represent the sequence of the desired membrane protein gene. The PCR fragment can then be used to isolate a full length cDNA clone by a variety of methods. For example, the amplified fragment can be labeled and used to screen a cDNA library, such as a bacteriophage cDNA library. Alternatively, the labeled fragment can be used to isolate genomic clones via the screening of a genomic library.

PCR technology can also be used to isolate full length cDNA sequences. For example, RNA can be isolated, following standard procedures, from an appropriate cellular or tissue source (i.e., one known to express, or suspected of expressing, a membrane protein gene). A reverse transcription (RT) reaction can be performed on the RNA using an oligonucleotide primer specific for the most 5′ end of the amplified fragment for the priming of first strand synthesis. The resulting RNA/DNA hybrid may then be “tailed” using a standard terminal transferase reaction, the hybrid may be digested with RNase H, and second strand synthesis may then be primed with a complementary primer. Thus, cDNA sequences upstream of the amplified fragment can be isolated. For a review of cloning strategies that can be used, see, e.g., “Molecular Cloning, A Laboratory Manual”, supra.

A cDNA encoding a mutant membrane protein sequence can be isolated, for example, by using PCR. In this case, the first cDNA strand may be synthesized by hybridizing an oligo-dT oligonucleotide to mRNA isolated from tissue known to express, or suspected of expressing, a normal version of the membrane protein, in an individual putatively carrying a mutant membrane protein allele, and by extending the new strand with reverse transcriptase. The second strand of the cDNA is then synthesized using an oligonucleotide that hybridizes specifically to the 5′ end of the normal sequence. Using these two primers, the product is then amplified via PCR, optionally cloned into a suitable vector, and subjected to DNA sequence analysis through methods well-known to those of skill in the art. By comparing the DNA sequence of the mutant membrane protein allele to that of a corresponding normal membrane protein allele, the mutation(s) responsible for the loss or alteration of function of the mutant membrane protein gene product can be ascertained.

Alternatively, a genomic library can be constructed using DNA obtained from an individual suspected of carrying, or known to carry, a mutant membrane protein allele (e.g., a person manifesting a membrane protein-associated phenotype such as, for example, osteoporosis, behavioral disorders, depression, colitis or spastic colon, obesity, high or low blood pressure, connective tissue disorders, hyperthyroidism or hypothyroidism, paralysis or palsy, nerve damage or degeneration, an inflammatory disorder, vision disorders, infertility, etc.), or a cDNA library can be constructed using RNA from a tissue known to express, or suspected of expressing, a mutant membrane protein allele. A normal membrane protein gene, or any suitable fragment thereof, can then be labeled and used as a probe to identify the corresponding mutant membrane protein allele in such libraries. Clones containing mutant membrane protein sequences can then be purified and subjected to sequence analysis according to methods well-known to those skilled in the art.

Additionally, an expression library can be constructed utilizing cDNA synthesized from, for example, RNA isolated from a tissue known to express, or suspected of expressing, a mutant membrane protein allele in an individual suspected of carrying, or known to carry, such a mutant allele. In this manner, gene products made by the putatively mutant tissue can be expressed and screened using standard antibody screening techniques in conjunction with antibodies raised against a normal membrane protein product, as described below (for screening techniques, see, for example, “Antibodies: A Laboratory Manual” (Harlow and Lane, eds., Cold Spring Harbor Press, Cold Spring Harbor, N.Y., 1988)).

Additionally, screening can be accomplished by screening with labeled membrane protein fusion proteins, such as, for example, alkaline phosphatase-membrane protein or membrane protein-alkaline phosphatase fusion proteins. In cases where a membrane protein mutation results in an expression product with altered function (e.g., as a result of a missense or a frameshift mutation), polyclonal antibodies to a membrane protein are likely to cross-react with a corresponding mutant membrane protein expression product. Library clones detected via their reaction with such labeled antibodies can be purified and subjected to sequence analysis according to methods well-known in the art.

The invention also encompasses: (a) DNA vectors that contain any of the foregoing membrane protein coding sequences and/or their complements (i.e., antisense); (b) DNA expression vectors that contain any of the foregoing membrane protein coding sequences operatively associated with a regulatory element that directs the expression of the coding sequences (for example, baculovirus as described in U.S. Pat. No. 5,869,336); (c) genetically engineered host cells that contain any of the foregoing membrane protein coding sequences operatively associated with a regulatory element that directs the expression of the coding sequences in the host cell; and (d) genetically engineered host cells that express an endogenous membrane protein sequence under the control of an exogenously introduced regulatory element (i.e., gene activation). As used herein, regulatory elements include, but are not limited to, inducible and non-inducible promoters, enhancers, operators, and other elements known to those skilled in the art that drive and regulate expression. Such regulatory elements include, but are not limited to, the cytomegalovirus (hCMV) immediate early gene, regulatable, viral elements (particularly retroviral LTR promoters), the early or late promoters of SV40 or adenovirus, the lac system, the trp system, the TAC system, the TRC system, the major operator and promoter regions of phage lambda, the control regions of fd coat protein, the promoter for 3-phosphoglycerate kinase (PGK), the promoters of acid phosphatase, and the promoters of the yeast α-mating factors.

The present invention also encompasses antibodies and anti-idiotypic antibodies (including Fab fragments), antagonists and agonists of a membrane protein, as well as compounds or nucleotide constructs that inhibit expression of a membrane protein sequence (transcription factor inhibitors, antisense and ribozyme molecules, or open reading frame sequence or regulatory sequence replacement constructs), or promote the expression of a membrane protein (e.g., expression constructs in which membrane protein coding sequences are operatively associated with expression control elements such as promoters, promoter/enhancers, etc.).

The membrane proteins, polypeptides, peptides, fusion proteins, nucleotide sequences, antibodies, antagonists and agonists can be useful for the detection of mutant membrane proteins, or inappropriately expressed membrane proteins, for the diagnosis of disease. The membrane proteins, peptides, fusion proteins, nucleotide sequences, host cell expression systems, antibodies, antagonists, agonists, and genetically engineered cells and animals can be used for screening for drugs (or high throughput screening of combinatorial libraries) effective in the treatment of the symptomatic or phenotypic manifestations of perturbing the normal function of a membrane protein in the body. The use of engineered host cells and/or animals may offer an advantage in that such systems allow not only for the identification of compounds that bind to the endogenous receptor for a membrane protein, but can also identify compounds that trigger membrane protein-mediated activities or pathways.

Finally, the described membrane protein products can be used as therapeutics. For example, soluble derivatives such as membrane protein peptides/domains, membrane protein fusion protein products (especially Ig fusion proteins, i.e., fusions of a membrane protein, or a domain of a membrane protein, to an IgFc), membrane protein antibodies and anti-idiotypic antibodies (including Fab fragments), antagonists or agonists (including compounds that modulate or act on downstream targets in a membrane protein-mediated pathway) can be used to directly treat diseases, including infectious diseases, or disorders. For instance, the administration of an effective amount of a soluble membrane protein product, a membrane protein-IgFc fusion protein, or an anti-idiotypic antibody (or its Fab) that mimics the membrane protein, could activate or effectively antagonize the endogenous membrane protein receptor. Nucleotide constructs encoding such membrane protein products can be used to genetically engineer host cells to express such products in vivo; these genetically engineered cells function as “bioreactors” in the body, delivering a continuous supply of a membrane protein, peptide, or fusion protein to the body. Nucleotide constructs encoding functional membrane proteins, mutant membrane proteins, as well as antisense and ribozyme molecules, can also be used in “gene therapy” approaches for the modulation of membrane protein expression. Thus, the invention also encompasses pharmaceutical formulations and methods for treating biological disorders.

Various aspects of the invention are described in greater detail in the subsections below.

### 7.1 Membrane Protein Nucleic Acid Sequences

The cDNA sequences and the corresponding deduced amino acid sequences of the described membrane proteins are presented in the Sequence Listing. The membrane protein nucleotide sequences were obtained from: gene trapped cDNAs, genomic sequence, and clones isolated from human brain, adipose, testis, and placenta cDNA libraries (SEQ ID NOS:1-7); clustered human gene trapped sequences, ESTs, and cDNA isolated from a human placenta cDNA cell library (SEQ ID NOS:8-12); analyzing human gene trapped sequence tags (SEQ ID NOS:13 and 14); analyzing human gene trapped sequence tags and cDNA clones isolated from a human kidney cDNA library (SEQ ID NOS:15-17); clustered human gene trapped sequences, genomic sequence, ESTs, and cDNAs from a human muscle cDNA library (SEQ ID NOS:18-45); clustered human gene trapped sequences, and cDNA products isolated from human brain and kidney mRNA (SEQ ID NOS:46-62); clustered human gene trapped sequences, and cDNA products isolated from human fetal brain and kidney libraries (SEQ ID NOS:63-95); clustered human gene trapped sequences, and cDNA products isolated from human skeletal muscle, colon, small intestine, fetal kidney, fetus, fetal brain, fetal lung, and mammary gland mRNAs (SEQ ID NOS:96-108); clustered genomic sequence, ESTs, and cDNAs produced using lung and testis mRNAs (SEQ ID NOS:109-112); clustered genomic sequence, ESTs, and cDNAs produced using human prostate, pituitary, skeletal muscle, fetal brain, brain, adrenal gland mRNAs (SEQ ID NOS:113-116); clustered genomic sequence, ESTs, and cDNAs produced using fetal kidney, kidney, and spinal cord mRNAs (SEQ ID NOS:117 and 118); clustered genomic sequence, ESTs, and cDNAs produced using fetal brain, fetal kidney, embryos, and embryonic carcinoma mRNAs (SEQ ID NOS:119-121); clustered genomic sequence, ESTs, and cDNAs produced using lymph node, hypothalamus, adrenal gland, lymph node, fetal kidney, adrenal gland, fetus, thyroid, uterus, and lung mRNAs (SEQ ID NOS:122-125); clustered genomic sequence, gene trapped sequences, ESTs, and cDNAs produced using placenta and brain mRNAs (SEQ ID NOS:126 and 127); cDNAs produced using lymph node and adrenal gland mRNAs (SEQ ID NOS:128 and 129); clustered genomic sequence, gene trapped sequences, ESTs, and cDNAs produced using fetal brain, fetal lung, pericardium, adrenal gland, lymph node, fetus, spinal cord, small intestine, mammary, colon, fetal kidney, lung, thyroid, prostate, and cerebellum mRNAs (SEQ ID NOS:130-132); clustered genomic sequence, gene trapped sequences, ESTs, and cDNAs produced using brain, cerebellum, hypothalamus, and kidney mRNAs (SEQ ID NOS:133 and 134); clustered genomic sequence, gene trapped sequences, ESTs, and cDNAs produced using adrenal gland, cerebellum, hypothalamus, fetal brain, brain and thyroid mRNAs (SEQ ID NOS:135 and 136); genomic sequence, and cDNA products isolated from human fetal brain, testis, and skeletal muscle mRNAs (SEQ ID NOS:137-148); clustered genomic sequence, gene trapped sequences, ESTs, and cDNAs produced from bone marrow and lymph node mRNAs (SEQ ID NOS:149-153); clustered genomic sequence, gene trapped sequences, ESTs, and cDNAs produced from bone marrow mRNAs (SEQ ID NOS:154-156); clustered ESTs, genomic sequence, and cDNAs generated from human lymph node, brain, spinal cord, and mammary gland mRNAs (SEQ ID NOS:157 and 158); and human genomic sequence and cDNAs made from human brain, adrenal gland, prostate, adipose, and testis mRNAs (SEQ ID NOS:159-163). The mRNAs and cDNA libraries were purchased from Clontech (Palo Alto, Calif.) and/or Edge Biosystems (Gaithersburg, Md.). Sequences corresponding to portions of certain of the presently described sequences have been described, and it is possible that such sequences were incomplete sequences that had been erroneously characterized as “full length”.

The genes encoding the described membrane proteins are located on: human chromosome 15, see GenBank Accession Numbers AC018900 and AK021660 (SEQ ID NOS:96-108); one or more of human chromosomes 10 and 11, see GenBank Accession Numbers AC084775 and AP002515 (SEQ ID NOS:109-112); human chromosome 5, see GenBank Accession Number AC026800 (SEQ ID NOS:113-116); human chromosome 9, see GenBank Accession Number AL445-489 (SEQ ID NOS:117 and 118); the human X chromosome, see GenBank Accession Number AB037734 (SEQ ID NOS:119-121); human chromosome 6, see GenBank Accession Number AL049553 (SEQ ID NOS:122-125); human chromosome 15, see GenBank Accession Number AC068507, which maps near a region associated with the autosomal recessive disorder Bardet-Biedl syndrome (SEQ ID NOS:126 and 127); human chromosome 3, see GenBank Accession Number AC024886 (SEQ ID NOS:128 and 129); human chromosome 4, see GenBank Accession Number AC027518 (SEQ ID NOS:130-132); human chromosome 15, see GenBank Accession Number AC022302(SEQ ID NOS:133 and 134); human chromosome 19, see GenBank Accession Number AC008761 (SEQ ID NOS:135 and 136); human chromosome 1, see GenBank Accession Number AL391234 (SEQ ID NOS:137-148); human chromosome 1, see GenBank Accession Number AL356276 (SEQ ID NOS:149-153); human chromosome 1, see GenBank Accession Number AL359970 (SEQ ID NOS:154-156); human chromosome 11, see GenBank Accession Number AP003501 (SEQ ID NOS:157 and 158); and human chromosome 1, see GenBank Accession Number AL121980 (SEQ ID NOS:159-163). As such, the described sequences can also be used to map the coding regions of the human genome, and to verify/identify mRNA exon splice junctions.

A variety polymorphisms were identified in the disclosed sequences, including: a translationally silent (“silent”) A/G polymorphism at nucleotide (“nt”) position 2106 of SEQ ID NO:8, and nt position 1827 of SEQ ID NO:10 (denoted by an “r” in the Sequence Listing), both of which result in a glutamine residue at corresponding amino acid (“aa”) position 702 of SEQ ID NO:9, and aa position 609 of SEQ ID NO:11, respectively; an A/C polymorphism at nt position 124 of SEQ ID NO:13 (denoted by an “m” in the Sequence Listing), which can result in an arginine or serine residue at corresponding aa position 42 of SEQ ID NO:14; a C/T polymorphism at nt position 233 of SEQ ID NO:13 (denoted by a “y” in the Sequence Listing), which can result in a threonine or methionine residue at corresponding aa position 78 of SEQ ID NO:14; a silent T/C polymorphism at nt position 1647 of SEQ ID NOS:18, 28, and 30, and nt position 822 of SEQ ID NOS:32, 42, and 44 (denoted by a “y” in the Sequence Listing), both of which result in a serine residue at corresponding aa position 549 of SEQ ID NOS:19, 29, and 31, and aa position 274 of SEQ ID NOS:33, 43, and 45, respectively; a G/C polymorphism at nt position 1884 of SEQ ID NOS:18 and 30, and nt position 1059 of SEQ ID NOS:32 and 44 (denoted by an “s” in the Sequence Listing), which can result in a leucine or phenylalanine residue at corresponding aa position 628 of SEQ ID NOS:19 and 31, and aa position 353 of SEQ ID NOS:33 and 45, respectively; an A/G polymorphism at nt position 2072 of SEQ ID NOS:18 and 30, and nt position 1247 of SEQ ID NOS:32 and 44 (denoted by an “r” in the Sequence Listing), which can result in a serine or asparagine residue at corresponding aa position 691 of SEQ ID NOS:19 and 31, and aa position 416 of SEQ ID NOS:33 and 45, respectively; an A/G polymorphism at nt position 2120 of SEQ ID NOS:18 and 30, and nt position 1295 of SEQ ID NOS:32 and 44 (denoted by an “r” in the Sequence Listing), which can result in an asparagine or serine residue at corresponding aa position 707 of SEQ ID NOS:19 and 31, and aa position 432 of SEQ ID NOS:33 and 45, respectively; a silent A/G polymorphism at nt position 2376 of SEQ ID NO:18, and nt position 1551 of SEQ ID NO:32 (denoted by an “r” in the Sequence Listing), both of which result in a proline residue at corresponding aa position 792 of SEQ ID NO:19, and aa position 517 of SEQ ID NO:33, respectively; an A/G polymorphism at nt position 2540 of SEQ ID NO:18, and nt position 1715 of SEQ ID NO:32 (denoted by an “r” in the Sequence Listing), which can result in a glutamate or glycine residue at corresponding aa position 847 of SEQ ID NO:19, and aa position 572 of SEQ ID NO:33, respectively; an A/G polymorphism at nt position 100 of SEQ ID NOS:46 and 50, nt position 232 of SEQ ID NOS:48 and 52, and nt position 406 of SEQ ID NOS:54 and 56 (denoted by an “r” in the sequence Listing), which can result in an asparagine or aspartate residue at corresponding aa position 34 of SEQ ID NOS:47 and 51, aa position 78 of SEQ ID NOS:49 and 53, and aa position 136 of SEQ ID NOS:55 and 57, respectively; a silent A/G polymorphism at nt position 792 of SEQ ID NOS:46 and 50, nt position 924 of SEQ ID NOS:48 and 52, nt position 1098 of SEQ ID NOS:54 and 56, and nt position 507 of SEQ ID NOS:58 and 60 (denoted by an “r” in the sequence Listing), both of which result in a proline residue at corresponding aa position 264 of SEQ ID NOS:47 and 51, aa position 308 of SEQ ID NOS:49 and 53, aa position 366 of SEQ ID NOS:55 and 57, and aa position 169 of SEQ ID NOS:59 and 61, respectively; a silent T/C polymorphism at nt position 840 of SEQ ID NOS:46 and 50, nt position 972 of SEQ ID NOS:48 and 52, nt position 1146 of SEQ ID NOS:54 and 56, and nt position 555 of SEQ ID NOS:58 and 60 (denoted by a “y” in the sequence Listing), both of which result in an isoleucine residue at corresponding aa position 280 of SEQ ID NOS:47 and 51, aa position 324 of SEQ ID NOS:49 and 53, aa position 382 of SEQ ID NOS:55 and 57, and aa position 185 of SEQ ID NOS:59 and 61, respectively; a GTG/null polymorphism at nt position 1974 of SEQ ID NOS:46 and 50, nt position 2106 of SEQ ID NOS:48 and 52, nt position 2280 of SEQ ID NOS:54 and 56, and nt position 1689 of SEQ ID NOS:58 and 60, which can result in a valine residue or no amino acid at corresponding aa position 658 of SEQ ID NOS:47 and 51, aa position 702 of SEQ ID NOS:49 and 53, aa position 760 of SEQ ID NOS:55 and 57, and aa position 563 of SEQ ID NOS:59 and 61, respectively; a silent C/T polymorphism at nt position 804 of SEQ ID NOS:65, 67, 69, 71, 73, 75, and 77, and nt position 507 of SEQ ID NOS:81, 83, 85, 87, 89, 91, and 93 (denoted by a “y” in the Sequence Listing), both of which result in an arginine residue at corresponding aa position 268 of SEQ ID NOS:66, 68, 70, 72, 74, 76, and 78, and aa position 169 of SEQ ID NOS:82, 84, 86, 88, 90, 92, and 94, respectively; a T/C polymorphism at nt position 2075 of SEQ ID NOS:71 and 75, nt position 2060 of SEQ ID NOS:73 and 77, nt position 1778 of SEQ ID NOS:87 and 91, and nt position 1763 of SEQ ID NOS:89 and 93 (denoted by a “y” in the Sequence Listing), which can give rise to a leucine or proline residue at corresponding aa position 692 of SEQ ID NOS:72 and 76, aa position 687 of SEQ ID NOS:74 and 78, aa position 593 of SEQ ID NOS:88 and 92, and aa position 588 of SEQ ID NOS:90 and 94, respectively; a T/A polymorphism at nt position 1108 of SEQ ID NOS:96, 98, and 100, and nt position 1069 of SEQ ID NOS:102, 104, and 106, which can result in a cysteine or serine residue at corresponding aa position 370 of SEQ ID NOS:97, 99, and 101, and aa position 357 of SEQ ID NOS:103, 105, and 107, respectively; a G/A polymorphism at nt position 1433 of SEQ ID NOS:96 and 98, and nt position 1394 of SEQ ID NOS:102 and 104, which can result in a serine or asparagine residue at corresponding aa position 478 of SEQ ID NOS:97 and 99, and aa position 465 of SEQ ID NOS:103 and 105, respectively; an A/G polymorphism at nt position 2366 of SEQ ID NO:96, nt position 2141 of SEQ ID NO:98, nt position 2327 of SEQ ID NO:102, and nt position 2102 of SEQ ID NO:104, which can result in a lysine or arginine residue at corresponding aa position 789 of SEQ ID NO:97, aa position 714 of SEQ ID NO:99, aa position 776 of SEQ ID NO:103, and aa position 701 of SEQ ID NO:105, respectively; a C/G polymorphism at nt position 241 of SEQ ID NOS:109 and 111, which can result in an arginine or glycine residue at corresponding aa position 81 of SEQ ID NOS:110 and 112; a C/G polymorphism at nt position 940 of SEQ ID NOS:109 and 111, which can result in a proline or alanine residue at corresponding aa position 314 of SEQ ID NOS:110 and 112; a silent C/T polymorphism at nt position 3465 of SEQ ID NOS:109 and 111, both of which result in an aspartate residue at corresponding aa position 1155 of SEQ ID NOS:110 and 112; a silent C/T polymorphism at nt position 5190 of SEQ ID NO:109, both of which result in a glycine residue at corresponding aa position 1730 of SEQ ID NO:110; an A/G polymorphism at nt position 1601 of SEQ ID NOS:113 and 115, which can result in a tyrosine or cysteine residue at corresponding aa position 534 of SEQ ID NOS:114 and 116; a T/G polymorphism at nt position 1704 of SEQ ID NOS:113 and 115, which can result in a cysteine or tryptophan residue at corresponding aa position 568 of SEQ ID NOS:114 and 116; a G/T polymorphism at nt position 1709 of SEQ ID NOS:113 and 115, which can result in a serine or isoleucine residue at corresponding aa position 600 of SEQ ID NOS:114 and 116; a T/G polymorphism at nt position 1712 of SEQ ID NOS:113 and 115, which can result in a valine or glycine residue at corresponding aa position 601 of SEQ ID NOS:114 and 116; a G/C polymorphism at nt position 631 of SEQ ID NO:117, which can result in an alanine or proline residue at corresponding aa position 211 of SEQ ID NO:118; a C/T polymorphism at nt position 2126 of SEQ ID NO:117, which can result in an alanine or valine residue at corresponding aa position 709 of SEQ ID NO:118; a G/A polymorphism at nt position 2422 of SEQ ID NO:117, which can result in a glutamate or lysine residue at corresponding aa position 808 of SEQ ID NO:118; a C/A polymorphism at nt position 2427 of SEQ ID NO:117, which can result in a glutamate or aspartate residue at corresponding aa position 809 of SEQ ID NO:118; a G/A polymorphism at nt position 2905 of SEQ ID NO:117, which can result in an alanine or threonine residue at corresponding aa position 969 of SEQ ID NO:118; a C/G polymorphism at nt position 2985 of SEQ ID NO:117, which can result in a phenylalanine or leucine residue at corresponding aa position 995 of SEQ ID NO:118; a T/C polymorphism at nt position 3329 of SEQ ID NO:117, which can result in a methionine or threonine residue at corresponding aa position 1110 of SEQ ID NO:118; a silent G/A polymorphism at nt position 489 of SEQ ID NO:122 (denoted by an “r” in the Sequence Listing), both of which result in a proline residue at corresponding aa position 163 of SEQ ID NO:123; a silent G/C polymorphism at nt position 1497 of SEQ ID NO:122 (denoted by an “s” in the Sequence Listing), both of which result in a leucine residue at corresponding aa position 499 of SEQ ID NO:123; a silent T/C polymorphism at nt position 345 of SEQ ID NO:128 (denoted by a “y” in the Sequence Listing), both of which result in an asparagine residue at corresponding aa position 115 of SEQ ID NO:129; a G/T polymorphism at nt position 448 of SEQ ID NO:128 (denoted by a “k” in the Sequence Listing), which can result in an aspartate or tyrosine residue at corresponding aa position 150 of SEQ ID NO:129; a silent G/A polymorphism at nt position 1347 of SEQ ID NO:128 (denoted by an “r” in the Sequence Listing), both of which result in a glutamine residue at corresponding aa position 449 of SEQ ID NO:129; a silent G/A polymorphism at nt position 2091 of SEQ ID NO:128 (denoted by an “r” in the Sequence Listing), both of which result in a glutamine residue at corresponding aa position 697 of SEQ ID NO:129; a silent T/C polymorphism at nt position 6729 of SEQ ID NO:128 (denoted by a “y” in the Sequence Listing), both of which result in an asparagine residue at corresponding aa position 2243 of SEQ ID NO:129; a silent G/A polymorphism at nt position 7683 of SEQ ID NO:128 (denoted by an “r” in the Sequence Listing), both of which result in a threonine residue at corresponding aa position 2561 of SEQ ID NO:129; a silent C/T polymorphism at nt position 405 of SEQ ID NO:130, both of which result in a phenylalanine residue at corresponding aa position 135 of SEQ ID NO:131; an A/T polymorphism at nt position 1358 of SEQ ID NO:130, which can result in a glutamine or leucine residue at corresponding aa position 453 of SEQ ID NO:131; a silent T/C polymorphism at nt position 2142 of SEQ ID NO:130, both of which result in a threonine residue at corresponding aa position 714 of SEQ ID NO:131; an A/G polymorphism at nt position 2267 of SEQ ID NO:130, which can result in a lysine or arginine residue at corresponding aa position 756 of SEQ ID NO:131; a silent T/A polymorphism at nt position 3429 of SEQ ID NO:130, both of which result in a leucine residue at corresponding aa position 1143 of SEQ ID NO:131; an A/G polymorphism at nt position 3628 of SEQ ID NO:130, which can result in a threonine or alanine residue at corresponding aa position 1210 of SEQ ID NO:131; a silent C/G polymorphism at nt position 4296 of SEQ ID NO:130, both of which result in a valine residue at corresponding aa position 1432 of SEQ ID NO:131; a silent A/G polymorphism at nt position 5799 of SEQ ID NO:130, both of which result in a proline residue at corresponding aa position 1933 of SEQ ID NO:131; an A/C polymorphism at nt position 6775 of SEQ ID NO:130, which can result in a threonine or proline residue at corresponding aa position 2259 of SEQ ID NO:131; a silent C/T polymorphism at nt position 6819 of SEQ ID NO:130, both of which result in an isoleucine residue at corresponding aa position 2273 of SEQ ID NO:131; a T/C polymorphism at nt position 7685 of SEQ ID NO:130, which can result in an isoleucine or threonine residue at corresponding aa position 2562 of SEQ ID NO:131; an A/G polymorphism at nt position 7960 of SEQ ID NO:130, which can result in an isoleucine or valine residue at corresponding aa position 2654 of SEQ ID NO:131; a C/G polymorphism at nt position 7981 of SEQ ID NO:130, which can result in a valine or leucine residue at corresponding aa position 2661 of SEQ ID NO:131; an A/G polymorphism at nt position 7991 of SEQ ID NO:130, which can result in a glutamate or glycine residue at corresponding aa position 2664 of SEQ ID NO:131; an A/G polymorphism at nt position 8213 of SEQ ID NO:130, which can result in an asparagine or serine residue at corresponding aa position 2738 of SEQ ID NO:131; a T/A polymorphism at nt position 11007 of SEQ ID NO:130, which can result in a serine or arginine residue at corresponding aa position 3669 of SEQ ID NO:131; a silent C/T polymorphism at nt position 11010 of SEQ ID NO:130, both of which result in an isoleucine residue at corresponding aa position 3670 of SEQ ID NO:131; a G/T polymorphism at nt position 11012 of SEQ ID NO:130, which can result in a cysteine or phenylalanine residue at corresponding aa position 3671 of SEQ ID NO:131; a silent G/A polymorphism at nt position 11193 of SEQ ID NO:130, both of which result in an arginine residue at corresponding aa position 3731 of SEQ ID NO:131; a C/T polymorphism at nt position 11197 of SEQ ID NO:130, which can result in a proline or cysteine residue at corresponding aa position 3733 of SEQ ID NO:131; an A/C polymorphism at nt position 11389 of SEQ ID NO:130, which can result in an asparagine or histidine residue at corresponding aa position 3797 of SEQ ID NO:131; a TT/CC polymorphism at nt positions 11399-11400 of SEQ ID NO:130, which can result in an asparagine or isoleucine residue at corresponding aa position 3800 of SEQ ID NO:131; a T/G polymorphism at nt position 11443 of SEQ ID NO:130, which can result in a leucine or valine residue at corresponding aa position 3814 of SEQ ID NO:131; a C/T polymorphism at nt position 11509 of SEQ ID NO:130, which can result in a proline or serine residue at corresponding aa position 3837 of SEQ ID NO:131; a C/T polymorphism at nt position 3235 of SEQ ID NO:133 (denoted by a “y” in the Sequence Listing), which can result in a histidine or tyrosine residue at corresponding aa position 1079 of SEQ ID NO:134; an A/T polymorphism at nt position 1075 of SEQ ID NO:135, which can result in an alanine or threonine residue at corresponding aa position 359 of SEQ ID NO:136; a G/A polymorphism at nt position 176 of SEQ ID NOS:137 and 139, nt position 14 of SEQ ID NOS:141 and 143, and nt position 59 of SEQ ID NOS:145 and 147, which can result in an arginine or glutamine residue at corresponding aa position 59 of SEQ ID NOS:138 and 140, aa position 5 of SEQ ID NOS:142 and 144, and aa position 20 of SEQ ID NOS:146 and 148, respectively; a G/A polymorphism at nt position 977 of SEQ ID NOS:137 and 139, nt position 815 of SEQ ID NOS:141 and 143, and nt position 860 of SEQ ID NOS:145 and 147, which can result in a glycine or glutamate residue at corresponding aa position 326 of SEQ ID NOS:138 and 140, aa position 272 of SEQ ID NOS:142 and 144, and aa position 287 of SEQ ID NOS:146 and 148, respectively; a C/A polymorphism at nt position 5598 of SEQ ID NO:137, nt position 5403 of SEQ ID NO:139, nt position 5436 of SEQ ID NO:141, nt position 5241 of SEQ ID NO:143, nt position 5481 of SEQ ID NO:145, and nt position 5286 of SEQ ID NO:147, which can result in a phenylalanine or leucine residue at corresponding aa position 1866 of SEQ ID NO:138, aa position 1801 of SEQ ID NO:140, aa position 1812 of SEQ ID NO:142, aa position 1747 of SEQ ID NO:144, aa position 1827 of SEQ ID NO:146, and aa position 1762 of SEQ ID NO:148, respectively; an A/G polymorphism at nt position 82 of SEQ ID NOS:149 and 151, which can result in an asparagine or aspartate residue at corresponding aa position 28 of SEQ ID NOS:150 and 152; a C/G polymorphism at nt position 210 of SEQ ID NOS:149 and 151, which can result in an aspartate or glutamate residue at corresponding aa position 70 of SEQ ID NOS:150 and 152; a C/A polymorphism at nt position 263 of SEQ ID NOS:149 and 151, which can result in a serine or tyrosine residue at corresponding aa position 88 of SEQ ID NOS:150 and 152; an A/G polymorphism at nt position 379 of SEQ ID NOS:149 and 151, which can result in an asparagine or aspartate at corresponding aa position 127 of SEQ ID NOS:150 and 152; a silent A/G polymorphism at nt position 390 of SEQ ID NOS:149 and 151, both of which result in a glutamine residue at corresponding aa position 130 of SEQ ID NOS:150 and 152; a silent T/C polymorphism at nt position 432 of SEQ ID NOS:149 and 151, both of which result in a tyrosine residue at corresponding aa position 144 of SEQ ID NOS:150 and 152; a C/A polymorphism at nt position 574 of SEQ ID NO:149, which can result in a proline or threonine residue at corresponding aa position 192 of SEQ ID NO:150; a silent C/T polymorphism at nt position 741 of SEQ ID NO:149, both of which result in an isoleucine residue at corresponding aa position 247 of SEQ ID NO:150; an A/T polymorphism at nt position 790 of SEQ ID NO:149, which can result in a threonine or serine residue at corresponding aa position 264 of SEQ ID NO:150; an A/G polymorphism at nt position 800 of SEQ ID NO:149, which can result in a histidine or arginine residue at corresponding aa position 267 of SEQ ID NO:150; a G/A polymorphism at nt position 886 of SEQ ID NO:149, which can result in a glycine or arginine residue at corresponding aa position 296 of SEQ ID NO:150; a CTG- or -GTC- or -GTG polymorphism at nt positions 895-897 of SEQ ID NO:149, which can result in a leucine or valine residue at corresponding aa position 299 of SEQ ID NO:150; an A/G polymorphism at nt position 968 of SEQ ID NO:149, which can result in a histidine or arginine at corresponding aa position 323 of SEQ ID NO:150; a silent A/C polymorphism at nt position 984 of SEQ ID NO:149, both of which result in a valine residue at corresponding aa position 328 of SEQ ID NO:150; a G/T polymorphism at nt position 994 of SEQ ID NO:149, which can result in a glycine or cysteine residue at corresponding aa position 332 of SEQ ID NO:150; a G/C polymorphism at nt position 998 of SEQ ID NO:149, which can result in an arginine or threonine residue at corresponding aa position 333 of SEQ ID NO:150; a silent G/A polymorphism at nt position 1047 of SEQ ID NO:149, both of which result in a lysine residue at corresponding aa position 349 of SEQ ID NO:150; a G/T polymorphism at nt position 1050 of SEQ ID NO:149, which can result in a glutamate or aspartate residue at corresponding aa position 350 of SEQ ID NO:150; an A/T polymorphism at nt position 1091 of SEQ ID NO:149, which can result in a histidine or leucine residue at corresponding aa position 364 of SEQ ID NO:150; a C/G polymorphism at nt position 1157 of SEQ ID NO:149, which can result in a threonine or serine residue at corresponding aa position 386 of SEQ ID NO:150; a silent C/G polymorphism at nt position 1218 of SEQ ID NO:149, both of which result in a serine residue at corresponding aa position 406 of SEQ ID NO:150; a G/T polymorphism at nt position 1260 of SEQ ID NO:149, which can result in a glutamate or aspartate residue at corresponding aa position 420 of SEQ ID NO:150; a G/T polymorphism at nt position 1285 of SEQ ID NO:149, which can result in an alanine or serine at corresponding aa position 429 of SEQ ID NO:150; an A/G polymorphism at nt position 1313 of SEQ ID NO:149, which can result in an asparagine or serine residue at corresponding aa position 438 of SEQ ID NO:150; a silent A/C polymorphism at nt position 1341 of SEQ ID NO:149, both of which result in a glycine residue at corresponding aa position 447 of SEQ ID NO:150; a silent T/C polymorphism at nt position 1509 of SEQ ID NO:149, both of which result in a serine residue at corresponding aa position 503 of SEQ ID NO:150; a CC/TT polymorphism at nt positions 1510-1511 of SEQ ID NO:149, which can result in a proline or phenylalanine residue at corresponding aa position 504 of SEQ ID NO:150; a CGA/TGG polymorphism at nt positions 1525-1527 of SEQ ID NO:149, which can result in an arginine or tryptophan residue at corresponding aa position 509 of SEQ ID NO:150; a C/A polymorphism at nt position 1639 of SEQ ID NO:149, which can result in a histidine or asparagine residue at corresponding aa position 547 of SEQ ID NO:150; a silent C/G polymorphism at nt position 1695 of SEQ ID NO:149, both of which result in a proline residue at corresponding aa position 565 of SEQ ID NO:150; a G/C polymorphism at nt position 1737 of SEQ ID NO:149, which can result in a glutamine or histidine residue at corresponding aa position 579 of SEQ ID NO:150; a G/T polymorphism at nt position 1774 of SEQ ID NO:149, which can result in an alanine or serine residue at corresponding aa position 592 of SEQ ID NO:150; a silent G/A polymorphism at nt position 1794 of SEQ ID NO:149, both of which result in a proline residue at corresponding aa position 598 of SEQ ID NO:150; a TGG/CGA polymorphism at nt positions 1804-1806 of SEQ ID NO:149, which can result in a tryptophan or arginine residue at corresponding aa position 602 of SEQ ID NO:150; a silent C/T polymorphism at nt position 1923 of SEQ ID NO:149, both of which result in an asparagine residue at corresponding aa position 641 of SEQ ID NO:150; a CG/TG/CA polymorphism at nt positions 1939-1940 of SEQ ID NO:149, which can result in an arginine, cysteine, or histidine residue at corresponding aa position 647 of SEQ ID NO:150; a G/C polymorphism at nt position 272 of SEQ ID NO:154, which can result in an arginine or proline residue at corresponding aa position 91 of SEQ ID NO:155; a G/C polymorphism at nt position 284 of SEQ ID NO:154, which can result in an arginine or proline residue at corresponding aa position 95 of SEQ ID NO:155; a T/C polymorphism at nt position 799 of SEQ ID NO:154, which can result in a tyrosine or histidine residue at corresponding aa position 267 of SEQ ID NO:155; a C/G polymorphism at nt position 1159 of SEQ ID NO:154, which can result in a proline or alanine residue at corresponding aa position 387 of SEQ ID NO:155; a silent C/G polymorphism at nt position 1224 of SEQ ID NO:154, both of which result in a leucine residue at corresponding aa position 408 of SEQ ID NO:155; a C/T polymorphism at nt position 1243 of SEQ ID NO:154, which can result in a histidine or tyrosine residue at corresponding aa position 415 of SEQ ID NO:155; a G/A polymorphism at nt position 1253 of SEQ ID NO:154, which can result in a glycine or aspartate residue at corresponding aa position 418 of SEQ ID NO:155; a silent T/C polymorphism at nt position 1269 of SEQ ID NO:154, both of which result in an arginine residue at corresponding aa position 423 of SEQ ID NO:155; a G/A polymorphism at nt position 1271 of SEQ ID NO:154, which can result in an arginine or lysine residue at corresponding aa position 424 of SEQ ID NO:155; a silent C/A polymorphism at nt position 1395 of SEQ ID NO:154, both of which result in a serine residue at corresponding aa position 465 of SEQ ID NO:155; a G/A polymorphism at nt position 1396 of SEQ ID NO:154, which can result in a valine or isoleucine residue at corresponding aa position 466 of SEQ ID NO:155; a silent C/T polymorphism at nt position 1404 of SEQ ID NO:154, both of which result in a valine residue at corresponding aa position 468 of SEQ ID NO:155; a silent T/C polymorphism at nt position 1407 of SEQ ID NO:154, both of which result in a proline residue at corresponding aa position 469 of SEQ ID NO:155; a silent A/C polymorphism at nt position 1668 of SEQ ID NO:154, both of which result in a serine residue at corresponding aa position 556 of SEQ ID NO:155; a G/A polymorphism at nt position 652 of SEQ ID NO:157 (denoted by an “r” in the Sequence Listing), which can result in a valine or methionine residue at corresponding aa position 218 of SEQ ID NO:158; and a silent G/A polymorphism at nt position 942 of SEQ ID NO:157 (denoted by an “r” in the Sequence Listing), both of which result in a lysine residue at corresponding aa position 314 of SEQ ID NO:158. The present invention contemplates sequences having any or all of the described polymorphisms, as well as all combinations and permutations thereof.

Because of their potential medical significance and their role in neural development, semaphorin protein family members have been subject to considerable scientific scrutiny. For example, U.S. Pat. Nos. 5,981,222, 6,013,781, and 5,935,865 describe other semaphorin protein family members, as well as applications, utilities, and uses that also pertain to the presently described semphorin-like proteins and semaphorin protein family members.

An additional application of the described novel human polynucleotide sequences is their use in the molecular mutagenesis/evolution of proteins that are at least partially encoded by the described novel sequences using, for example, polynucleotide shuffling or related methodologies. Such approaches are described in U.S. Pat. Nos. 5,830,721 and 5,837,458.

The described membrane protein gene products can also be expressed in non-human transgenic animals. Animals of any non-human species, including, but not limited to, worms, mice, rats, rabbits, guinea pigs, pigs, micro-pigs, birds, goats, and non-human primates, e.g., baboons, monkeys, and chimpanzees, may be used to generate membrane protein transgenic animals.

Any technique known in the art may be used to introduce a membrane protein transgene into animals to produce the founder lines of transgenic animals. Such techniques include, but are not limited to, pronuclear microinjection (U.S. Pat. No. 4,873,191), retrovirus-mediated gene transfer into germ lines (Van der Putten et al., Proc. Natl. Acad. Sci. USA 82:6148-6152, 1985), gene targeting in embryonic stem cells (Thompson et al., Cell 56:313-321, 1989), electroporation of embryos (Lo, Mol. Cell. Biol. 3:1803-1814, 1983), and sperm-mediated gene transfer (Lavitrano et al., Cell 57:717-723, 1989), etc. For a review of such techniques, see, e.g., Gordon, Intl. Rev. Cytol. 115:171-229, 1989.

The present invention provides for transgenic animals that carry a membrane protein transgene in all their cells, as well as animals that carry a membrane protein transgene in some, but not all their cells, i.e., mosaic animals or somatic cell transgenic animals. A transgene may be integrated as a single transgene, or in concatamers, e.g., head-to-head tandems or head-to-tail tandems. A transgene may also be selectively introduced into and activated in a particular cell-type by following, for example, the teaching of Lasko et al., Proc. Natl. Acad. Sci. USA 89:6232-6236, 1992. The regulatory sequences required for such a cell-type specific activation will depend upon the particular cell-type of interest, and will be apparent to those of skill in the art.

When it is desired that a membrane protein transgene be integrated into the chromosomal site of the endogenous membrane protein gene, gene targeting is preferred. Briefly, when such a technique is to be utilized, vectors containing some nucleotide sequences homologous to the endogenous membrane protein gene are designed for the purpose of integrating, via homologous recombination with chromosomal sequences, into and disrupting the function of the nucleotide sequence of the endogenous membrane protein gene (i.e., “knock-out” animals).

The transgene can also be selectively introduced into a particular cell-type, thus inactivating the endogenous membrane protein gene in only that cell-type, by following, for example, the teaching of Gu et al., Science 265:103-106, 1994. The regulatory sequences required for such a cell-type specific inactivation will depend upon the particular cell-type of interest, and will be apparent to those of skill in the art.

Once transgenic animals have been generated, the expression of the recombinant membrane protein gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques that include, but are not limited to, Northern blot analysis of tissue samples obtained from the animal, in situ hybridization analysis, and RT-PCR. Samples of membrane protein gene-expressing tissue may also be evaluated immunocytochemically using antibodies specific for a membrane protein transgene product.

The present invention also provides for “knock-in” animals. Knock-in animals are those in which a polynucleotide sequence (i.e., a gene or a cDNA) that the animal does not naturally have in its genome is inserted in such a way that it is expressed. Examples include, but are not limited to, a human gene or cDNA used to replace its murine ortholog in the mouse, a murine cDNA used to replace the murine gene in the mouse, and a human gene or cDNA or murine cDNA that is tagged with a reporter construct used to replace the murine ortholog or gene in the mouse. Such replacements can occur at the locus of the murine ortholog or gene, or at another specific site. Such knock-in animals are useful for the in vivo study, testing and validation of, intra alia, human drug targets, as well as for compounds that are directed at the same, and therapeutic proteins.

### 7.2 Membrane Protein Amino Acid Sequences

The described membrane proteins, polypeptides, peptide fragments, mutated, truncated, or deleted forms of the membrane proteins, and/or membrane protein fusion proteins can be prepared for a variety of uses. These uses include, but are not limited to, the generation of antibodies, as reagents in diagnostic assays, for the identification of other cellular gene products related to the described membrane proteins, and as reagents in assays for screening for compounds that can be used as pharmaceutical reagents useful in the therapeutic treatment of mental, biological, or medical disorders and diseases. Given the similarity information and expression data, the described membrane proteins can be targeted (by drugs, oligonulceotides, antibodies, etc.) in order to treat disease (and particularly diseases of the blood or immune system), to therapeutically supplant or augment the efficacy of, for example, chemotherapeutic agents used in the treatment of cancer (for example breast or prostate cancer), agents used to promote healing or in the treatment of inflammatory disorders, arthritis, or infectious disease, or as antiviral agents. The described sequences are also useful as cancer, particularly carcinoma, diagnostic markers.

The Sequence Listing discloses the amino acid sequences encoded by the described membrane protein nucleic acid sequences. The membrane proteins display initiator methionines in DNA sequence contexts consistent with translation initiation sites, and many display hydrophobic regions near their N-termini that can serve as signal sequences, which indicates that the described membrane proteins can be membrane-associated, secreted, or cytoplasmic. Certain of the described proteins have tandem N-terminal methionines, either of which can serve to initiate translation. Of particular note, solubilized versions of the N-terminal extracellular domain(s) of SEQ ID NOS:123 and 125 (approximately 170 N-terminal amino acids less the signal sequence) can be directly used in therapeutic applications.

The amino acid sequences of the invention include the amino acid sequence presented in the Sequence Listing, as well as analogues and derivatives thereof. Further, corresponding membrane protein homologues from other species are encompassed by the invention. In fact, any membrane protein encoded by the membrane protein nucleotide sequences described herein are within the scope of the invention, as are any novel polynucleotide sequences encoding all or any novel portion of an amino acid sequence presented in the Sequence Listing. The degenerate nature of the genetic code is well-known, and, accordingly, each amino acid presented in the Sequence Listing is generically representative of the well-known nucleic acid “triplet” codon, or in many cases codons, that can encode the amino acid. As such, as contemplated herein, the amino acid sequences presented in the Sequence Listing, when taken together with the genetic code (see, for example, “Molecular Cell Biology”, Table 4-1 at page 109 (Darnell et al., eds., Scientific American Books, New York, N.Y., 1986)), are generically representative of all the various permutations and combinations of nucleic acid sequences that can encode such amino acid sequences.

The invention also encompasses proteins that are functionally equivalent to the membrane proteins encoded by the presently described nucleotide sequences, as judged by any of a number of criteria, including, but not limited to, the ability to bind and cleave a substrate of a membrane protein, the ability to effect an identical or complementary downstream pathway, or a change in cellular metabolism (e.g., proteolytic activity, ion flux, tyrosine phosphorylation, etc.). Such functionally equivalent membrane proteins include, but are not limited to, additions or substitutions of amino acid residues within the amino acid sequence encoded by the membrane protein nucleotide sequences described herein, but that result in a silent change, thus producing a functionally equivalent expression product. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

A variety of host-expression vector systems can be used to express the membrane protein nucleotide sequences of the invention. Where, as in the present instance, the described proteins are thought to be membrane proteins, the hydrophobic regions of the protein can be excised and the resulting soluble peptide or polypeptide can be recovered from the culture media. Such expression systems also encompass engineered host cells that express a membrane protein, or functional equivalent, in situ. Purification or enrichment of a membrane protein from such expression systems can be accomplished using appropriate detergents and lipid micelles and methods well-known to those skilled in the art. However, such engineered host cells themselves may be used in situations where it is important not only to retain the structural and functional characteristics of a membrane protein, but to assess biological activity, e.g., in certain drug screening assays.

The expression systems that may be used for purposes of the invention include, but are not limited to, microorganisms such as bacteria (e.g., E. coli, B. subtilis) transformed with recombinant bacteriophage DNA, plasmid DNA or cosmid DNA expression vectors containing membrane protein nucleotide sequences; yeast (e.g., Saccharomyces, Pichia) transformed with recombinant yeast expression vectors containing membrane protein nucleotide sequences; insect cell systems infected with recombinant virus expression vectors (e.g., baculovirus) containing membrane protein nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing membrane protein nucleotide sequences; or mammalian cell systems (e.g., COS, CHO, BHK, 293, 3T3) harboring recombinant expression constructs containing membrane protein nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the membrane protein product being expressed. For example, when a large quantity of such a protein is to be produced for the generation of pharmaceutical compositions of or containing a membrane protein, or for raising antibodies to a membrane protein, vectors that direct the expression of high levels of fusion protein products that are readily purified may be desirable. Such vectors include, but are not limited to, the E. coli expression vector pUR278 (Ruther and Muller-Hill, EMBO J. 2:1791-1794, 1983), in which a membrane protein coding sequence may be ligated individually into the vector in frame with the lacZ coding region so that a fusion protein is produced, pIN vectors (Inouye and Inouye, Nucl. Acids Res. 13:3101-3109, 1985; Van Heeke and Schuster, J. Biol. Chem. 264:5503-5509, 1989), and the like. pGEX vectors (Pharmacia or American Type Culture Collection) can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. A membrane protein coding sequence can be cloned individually into a non-essential region (for example the polyhedrin gene) of the virus and placed under control of an AcNPV promoter (for example the polyhedrin promoter). Successful insertion of a membrane protein coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (see, e.g., Smith et al., J. Virol. 46:584-593, 1983, and U.S. Pat. No. 4,215,051).

In mammalian host cells, a number of viral-based expression systems may be utilized. In cases where an adenovirus is used as an expression vector, the membrane protein nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a membrane protein product in infected hosts (see, e.g., Logan and Shenk, Proc. Natl. Acad. Sci. USA 81:3655-3659, 1984). Specific initiation signals may also be required for efficient translation of inserted membrane protein nucleotide sequences. These signals include the ATG initiation codon and adjacent sequences. In cases where an entire membrane protein gene or cDNA, including its own initiation codon and adjacent sequences, is inserted into the appropriate expression vector, no additional translational control signals may be needed. However, in cases where only a portion of a membrane protein coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bitter et al., Methods Enzymol. 153:516-544, 1987).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, WI38, and in particular, human cell lines.

For long-term, high-yield production of recombinant proteins, stable expression is preferred. For example, cell lines that stably express the membrane protein sequences described herein can be engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express a membrane protein product. Such engineered cell lines may be particularly useful in screening and evaluation of compounds that affect the endogenous activity of a membrane protein product.

A number of selection systems may be used, including, but not limited to, the herpes simplex virus thymidine kinase (Wigler et al., Cell 11:223-232, 1977), hypoxanthine-guanine phosphoribosyltransferase (Szybalska and Szybalski, Proc. Natl. Acad. Sci. USA 48:2026-2034, 1962), and adenine phosphoribosyltransferase (Lowy et al., Cell 22:817-823, 1980) genes, which can be employed in tk⁻, hgprt⁻ or aprt⁻ cells, respectively. Also, antimetabolite resistance can be used as the basis of selection for the following genes: dihydrofolate reductase (dhfr), which confers resistance to methotrexate (Wigler et al., Proc. Natl. Acad. Sci. USA 77:3567-3570, 1980, and O'Hare et al., Proc. Natl. Acad. Sci. USA 78:1527-1531, 1981); guanine phosphoribosyl transferase (gpt), which confers resistance to mycophenolic acid (Mulligan and Berg, Proc. Natl. Acad. Sci. USA 78:2072-2076, 1981); neomycin phosphotransferase (neo), which confers resistance to G-418 (Colbere-Garapin et al., J. Mol. Biol. 150:1-14, 1981); and hygromycin B phosphotransferase (hpt), which confers resistance to hygromycin (Santerre et al., Gene 30:147-156, 1984).

Alternatively, any fusion protein can be readily purified by utilizing an antibody specific for the fusion protein being expressed. Another exemplary system allows for the ready purification of non-denatured fusion proteins expressed in human cell lines (Janknecht et al., Proc. Natl. Acad. Sci. USA 88:8972-8976, 1991). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺-nitriloacetic acid-agarose columns, and histidine-tagged proteins are selectively eluted with imidazole-containing buffers.

Also encompassed by the present invention are fusion proteins that direct a membrane protein to a target organ and/or facilitate transport across the membrane into the cytosol. Conjugation of membrane proteins to antibody molecules or their Fab fragments could be used to target cells bearing a particular epitope. Attaching an appropriate signal sequence to a membrane protein would also transport a membrane protein to a desired location within the cell. Alternatively, targeting of a membrane protein or its nucleic acid sequence might be achieved using liposome or lipid complex based delivery systems. Such technologies are described in “Liposomes: A Practical Approach” (New, R. R. C., ed., IRL Press, New York, N.Y., 1990), and in U.S. Pat. Nos. 4,594,595, 5,459,127, 5,948,767 and 6,110,490. Additionally embodied are novel protein constructs engineered in such a way that they facilitate transport of membrane proteins to a target site or desired organ, where they cross the cell membrane and/or the nucleus, where the membrane proteins can exert their functional activity. This goal may be achieved by coupling of a membrane protein to a cytokine or other ligand that provides targeting specificity, and/or to a protein transducing domain (see generally U.S. Provisional Patent Application Serial Nos. 60/111,701 and 60/056,713 for examples of such transducing sequences), to facilitate passage across cellular membranes, and can optionally be engineered to include nuclear localization signals.

Additionally contemplated are oligopeptides that are modeled on an amino acid sequence first described in the Sequence Listing. Such membrane protein oligopeptides are generally between about 10 to about 100 amino acids long, or between about 16 to about 80 amino acids long, or between about 20 to about 35 amino acids long, or any variation or combination of sizes represented therein that incorporate a contiguous region of sequence first disclosed in the Sequence Listing. Such membrane protein oligopeptides can be of any length disclosed within the above ranges and can initiate at any amino acid position represented in the Sequence Listing.

The invention also contemplates “substantially isolated” or “substantially pure” proteins or polypeptides. By a “substantially isolated” or “substantially pure” protein or polypeptide is meant a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### 7.3 Antibodies to Membrane Proteins

Antibodies that specifically recognize one or more epitopes of a membrane protein, epitopes of conserved variants of a membrane protein, or peptide fragments of a membrane protein, are also encompassed by the invention. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of a membrane protein in a biological sample and may, therefore, be utilized as part of a diagnostic or prognostic technique whereby patients may be tested for abnormal amounts of a membrane protein. Such antibodies may also be utilized in conjunction with, for example, compound screening schemes for the evaluation of the effect of test compounds on expression and/or activity of a membrane protein expression product. Additionally, such antibodies can be used in conjunction with gene therapy to, for example, evaluate normal and/or engineered membrane protein-expressing cells prior to their introduction into a patient. Such antibodies may additionally be used in methods for the inhibition of abnormal membrane protein activity. Thus, such antibodies may be utilized as a part of treatment methods.

For the production of antibodies, various host animals may be immunized by injection with a membrane protein, a membrane protein peptide (e.g., one corresponding to a functional domain of a membrane protein), a truncated membrane protein polypeptide (a membrane protein in which one or more domains have been deleted), functional equivalents of a membrane protein or mutated variants of a membrane protein. Such host animals may include, but are not limited to, pigs, rabbits, mice, goats, and rats, to name but a few. Various adjuvants may be used to increase the immunological response, depending on the host species, including, but not limited to, Freund's adjuvant (complete and incomplete), mineral salts such as aluminum hydroxide or aluminum phosphate, chitosan, surface active substances such as lysolecithin, pluronic polyols, polyanions, peptides, oil emulsions, and potentially useful human adjuvants such as BCG (bacille Calmette-Guerin) and Corynebacterium parvum. Alternatively, the immune response could be enhanced by combination and/or coupling with molecules such as keyhole limpet hemocyanin, tetanus toxoid, diphtheria toxoid, ovalbumin, cholera toxin, or fragments thereof. Polyclonal antibodies are heterogeneous populations of antibody molecules derived from the sera of the immunized animals.

Monoclonal antibodies, which are homogeneous populations of antibodies to a particular antigen, can be obtained by any technique that provides for the production of antibody molecules by continuous cell lines in culture. These include, but are not limited to, the hybridoma technique (Kohler and Milstein, Nature 256:495-497, 1975, and U.S. Pat. No. 4,376,110), the human B-cell hybridoma technique (Kosbor et al., Immunology Today 4:72, 1983, and Cole et al., Proc. Natl. Acad. Sci. USA 80:2026-2030, 1983), and the EBV-hybridoma technique (Cole et al., in “Monoclonal Antibodies and Cancer Therapy”, Vol. 27, UCLA Symposia on Molecular and Cellular Biology, New Series, pp. 77-96 (Reisfeld and Sell, eds., Alan R. Liss, Inc. New York, N.Y., 1985)). Such antibodies may be of any immunoglobulin class, including IgG, IgM, IgE, IgA, and IgD, and any subclass thereof. The hybridomas producing the mabs of this invention may be cultivated in vitro or in vivo. Production of high titers of mAbs in vivo makes this the presently preferred method of production.

In addition, techniques developed for the production of “chimeric antibodies” (Morrison et al., Proc. Natl. Acad. Sci. USA 81:6851-6855, 1984, Neuberger et al., Nature 312:604-608, 1984, and Takeda et al., Nature 314:452-454, 1985) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region. Such technologies are described in U.S. Pat. Nos. 6,114,598, 6,075,181, and 5,877,397. Also encompassed by the present invention is the use of fully humanized monoclonal antibodies, as described in U.S. Pat. No. 6,150,584.

Alternatively, techniques described for the production of single chain antibodies (U.S. Pat. No. 4,946,778, Bird, Science 242:423-426, 1988, Huston et al., Proc. Natl. Acad. Sci. USA 85:5879-5883, 1988, and Ward et al., Nature 341:544-546, 1989) can be adapted to produce single chain antibodies against membrane protein expression products. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: F(ab′)₂ fragments, which can be produced by pepsin digestion of an antibody molecule; and Fab fragments, which can be generated by reducing the disulfide bridges of F(ab′)₂ fragments. Alternatively, Fab expression libraries may be constructed (Huse et al., Science 246:1275-1281, 1989) to allow rapid and easy identification of monoclonal Fab fragments with the desired specificity.

Antibodies to a membrane protein can, in turn, be utilized to generate anti-idiotype antibodies that “mimic” a given membrane protein, using techniques well-known to those skilled in the art (see, e.g., Greenspan and Bona, FASEB J. 7:437-444, 1993, and Nissinoff, J. Immunol. 147:2429-2438, 1991). For example, antibodies that bind to a membrane protein domain and competitively inhibit the binding of the membrane protein to its cognate receptor can be used to generate anti-idiotypes that “mimic” the membrane protein and, therefore, bind and activate or neutralize a receptor. Such anti-idiotypic antibodies, or Fab fragments of such anti-idiotypes, can be used in therapeutic regimens involving a membrane protein-mediated pathway.

Additionally given the high degree of relatedness of mammalian membrane proteins, membrane protein knock-out mice (having never seen a membrane protein, and thus never been tolerized to a membrane protein) have an unique utility, as they can be advantageously applied to the generation of antibodies against the disclosed mammalian membrane proteins (i.e., a membrane protein will be immunogenic in membrane protein knock-out animals).

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety.

1. An isolated nucleic acid molecule comprising a nucleotide sequence that encodes the amino acid sequence of SEQ ID NO:2, 4, 6, 9, 11, 14, 16, 19, 21, 23, 25, 27, 29, 31, 33, 35, 37, 39, 41, 43, 45, 47, 49, 51, 53, 55, 57, 59, 61, 64, 66, 68, 70, 72, 74, 76, 78, 80, 82, 84, 86, 88, 90, 92, 94, 97, 99, 101, 103, 105, 107, 110, 112, 114, 116, 118, 120, 123, 125, 127, 129, 131, 134, 136, 138, 140, 142, 144, 146, 148, 150, 152, 155, 158, 160, or 162.

2. The isolated nucleic acid molecule of claim 1, wherein said nucleic acid molecule comprises the nucleotide sequence of SEQ ID NO:1, 3, 5, 8, 10, 13, 15, 18, 20, 22, 24, 26, 28, 30, 32, 34, 36, 38, 40, 42, 44, 46, 48, 50, 52, 54, 56, 58, 60, 63, 65, 67, 69, 71, 73, 75, 77, 79, 81, 83, 85, 87, 89, 91, 93, 96, 98, 100, 102, 104, 106, 109, 111, 113, 115, 117, 119, 122, 124, 126, 128, 130, 133, 135, 137, 139, 141, 143, 145, 147, 149, 151, 154, 157, 159, or 161.

3. An expression vector comprising the isolated nucleic acid molecule of claim 1.

4. An isolated polypeptide comprising the amino acid sequence of SEQ ID NO:2, 4, 6, 9, 11, 14, 16, 19, 21, 23, 25, 27, 29, 31, 33, 35, 37, 39, 41, 43, 45, 47, 49, 51, 53, 55, 57, 59, 61, 64, 66, 68, 70, 72, 74, 76, 78, 80, 82, 84, 86, 88, 90, 92, 94, 97, 99, 101, 103, 105, 107, 110, 112, 114, 116, 118, 120, 123, 125, 127, 129, 131, 134, 136, 138, 140, 142, 144, 146, 148, 150, 152, 155, 158, 160, or 162.

5. The isolated polypeptide of claim 4, comprising the amino acid sequence of SEQ ID NO: 97, 99, 101, 103, 105, 107, 110, 118, 120, 123, 125, 131, 134, 136, 138, 140, 142, 144, 146, 148, or 158.

6. The isolated nucleic acid molecule of claim 1, wherein said nucleic acid molecule encodes the amino acid sequence of SEQ ID NO: 97, 99, 101, 103, 105, 107, 110, 118, 120, 123, 125, 131, 134, 136, 138, 140, 142, 144, 146, 148, or 158.

7. The isolated nucleic acid molecule of claim 1, wherein said nucleic acid molecule comprises the nucleotide sequence of SEQ ID NO: 96, 98, 100, 102, 104, 106, 117, 119, 122, 124, 130, 133, 135, 137, 139, 141, 143, 145, 147, or 157.

8. An oligonucleotide that inhibits the expression of a nucleic acid molecule that encodes an amino acid sequence of SEQ ID NO: 2, 4, 6, 9, 11, 14, 16, 19, 21, 23, 25, 27, 29, 31, 33, 35, 37, 39, 41, 43, 45, 47, 49, 51, 53, 55, 57, 59, 61, 64, 66, 68, 70, 72, 74, 76, 78, 80, 82, 84, 86, 88, 90, 92, 94, 97, 99, 101, 103, 105, 107, 110, 112, 114, 116, 118, 120, 123, 125, 127, 129, 131, 134, 136, 138, 140, 142, 144, 146, 148, 150, 152, 155, 158, 160, or 162.

9. The oligonucleotide of claim 8, wherein said nucleic acid molecule encodes the amino acid sequence of SEQ ID NO: 97, 99, 101, 103, 105, 107, 110, 118, 120, 123, 125, 131, 134, 136, 138, 140, 142, 144, 146, 148, or 158.

10. An antibody that selectively binds a polypeptide comprising an amino acid sequence of SEQ ID NOS: 2, 4, 6, 9, 11, 14, 16, 19, 21, 23, 25, 27, 29, 31, 33, 35, 37, 39, 41, 43, 45, 47, 49, 51, 53, 55, 57, 59, 61, 64, 66, 68, 70, 72, 74, 76, 78, 80, 82, 84, 86, 88, 90, 92, 94, 97, 99, 101, 103, 105, 107, 110, 112, 114, 116, 118, 120, 123, 125, 127, 129, 131, 134, 136, 138, 140, 142, 144, 146, 148, 150, 152, 155, 158, 160, or 162.

11. The antibody claim 10, wherein said polypeptide comprises the amino acid sequence of SEQ ID NO: 97, 99, 101, 103, 105, 107, 110, 118, 120, 123, 125, 131, 134, 136, 138, 140, 142, 144, 146, 148, or 158.
